(19) United States
(12) Patent Application Publication
(10) Pub. No.: US 2018/0186753 A1
(43) Pub. Date: Jul. 5, 2018

(54) PROCESSES FOR THE PREPARATION OF PESTICIDAL COMPOUNDS

(71) Applicant: Dow AgroSciences LLC, Indianapolis, IN (US)

(72) Inventors: Qiang YANG, Indianapolis, IN (US); Beth LORSBACH, Indianapolis, IN (US); Noormohamed M. NIYAZ, Indianapolis, IN (US); Ann M. BUYSSE, Indianapolis, IN (US); Martin J. WALSH, Indianapolis, IN (US); Yu Zhang, Indianapolis, IN (US); Tony K. TRULLINGER, Indianapolis, IN (US); Erich J. MOLITOR, Midland, MI (US); Belgin CANTURK, Indianapolis, IN (US); Yan HAO, Indianapolis, IN (US); Jeremy KISTER, Indianapolis, IN (US)

(21) Appl. No.: 15/853,073

(22) Filed: Dec. 22, 2017

Related U.S. Application Data
(60) Provisional application No. 62/440,227, filed on Dec. 29, 2016.

(51) Int. Cl. C07D 231/40; C07D 401/04

(52) U.S. Cl. CPC C07D 231/40; C07D 401/04

(57) ABSTRACT

The disclosure relates to efficient and economical synthetic chemical processes for the preparation of pesticidal thioethers. Further, the disclosure relates to certain novel compounds useful in the preparation of pesticidal thioethers.

### CROSS-REFERENCE TO RELATED APPLICATIONS

This application claims priority under 35 U.S.C. § 119(e) to U.S. Provisional Application Ser. No. 62/440,227 filed Dec. 29, 2016, which is incorporated herein by this reference in its entirety.

### TECHNICAL FIELD

This application relates to efficient and economical synthetic chemical processes for the preparation of pesticidal thioethers. Further, the present application relates to certain novel compounds useful in the preparation of pesticidal thioethers.

### BACKGROUND

There are more than ten thousand species of pests that cause losses in agriculture. The world-wide agricultural losses amount to billions of U.S. dollars each year. Stored food pests eat and adulterate stored food. The world-wide stored food losses amount to billions of U.S. dollars each year, but more importantly, deprive people of needed food. Certain pests have developed resistance to pesticides in current use. Hundreds of pest species are resistant to one or more pesticides. The development of resistance to some of the older pesticides, such as DDT, the carbamates, and the organophosphates, is well known. But resistance has even developed to some of the newer pesticides. As a result, there is an acute need for new pesticides that has led to the development of new pesticides. Specifically, US 20130288893(A1) describes, inter alia, certain pesticidal thioethers and their use as pesticides. Such compounds are finding use in agriculture for the control of pests.

Because there is a need for very large quantities of pesticides, specifically pesticidal thioethers, it would be advantageous to produce pesticidal thioethers efficiently and in high yield from commercially available starting materials to provide the market with more economical sources of much needed pesticides.

### DEFINITIONS

As used herein, the term “alkyl” includes a chain of carbon atoms, which is optionally branched including but not limited to C₁-C₆, C₁-C₄, and C₁-C₃. Illustrative alkyl groups include, but are not limited to, methyl, ethyl, n-propyl, isopropyl, n-butyl, isobutyl, sec-butyl, tert-butyl, pentyl, 2-pentyl, 3-pentyl, and the like. Alkyl may be substituted or unsubstituted. It will be understood that “alkyl” may be combined with other groups, such as those provided above, to form a functionalized alkyl. By way of example, the combination of an “alkyl” group, as described herein, with a “cycloalkyl” group may be referred to as an “alkyl-cycloalkyl” group.

As used herein, the term “cycloalkyl” refers to an all-carbon cyclic ring, optionally containing one or more double bonds but the cycloalkyl does not contain a completely conjugated pi-electron system. It will be understood that in certain embodiments, cycloalkyl may be advantageously of limited size, such as C₃-C₆. Cycloalkyl may be unsubstituted or substituted. Examples of cycloalkyl include cyclopropyl, cyclobutyl, and cyclohexyl.

As used herein, the term “aryl” refers to an all-carbon cyclic ring containing a completely conjugated pi-electron system. It will be understood that in certain embodiments, aryl may be advantageously of limited size, such as C₆-C₁₀. Aryl may be unsubstituted or substituted. Examples of aryl include phenyl and naphthyl.

As used herein, “halo” or “halogen” or “halide” may be used interchangeably and refers to fluorine (F), chlorine (Cl), bromine (Br) or iodine (I).

As used herein, “trihalomethyl” refers to a methyl group having three halo substituents, such as a trifluoromethyl group.

### DETAILED DESCRIPTION

This application relates to efficient and economical synthetic chemical processes for the preparation of pesticidal thioethers. Further, the present application relates to certain novel compounds useful in the preparation of pesticidal thioethers.

The compounds and process of the present application are described in detail below. The processes of the present disclosure can be described according to Scheme 1.

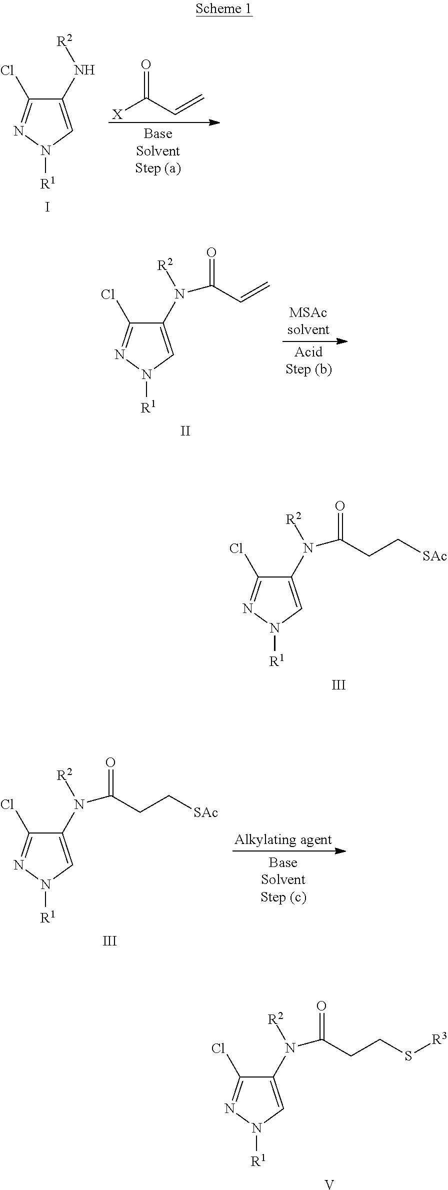

In Step (a) of Scheme 1, the compound of the formula I is acylated with an acryloyl reagent of the formula X—C(O)CH═CH₂, wherein X is a leaving group, such as a F, Cl, Br, I, —OC(O)C₁-C₆ alkyl, —OC(O)C₆-C₁₀ aryl, and the like, in the presence of a base. The base in Step (a) can be an inorganic base, such as sodium bicarbonate (NaHCO₃), sodium carbonate (Na₂CO₃), calcium carbonate (CaCO₃), cesium carbonate (Cs₂CO₃), lithium carbonate (Li₂CO₃), potassium carbonate (K₂CO₃), lithium hydroxide (LiOH), sodium hydroxide (NaOH), potassium hydroxide (KOH), cesium hydroxide (CsOH), calcium hydroxide (Ca(OH)₂), sodium diphosphate (Na₂HPO₄), potassium phosphate (K₃PO₄), and the like. Alternatively, the base in Step (a) can be an organic base, such as triethylamine (TEA), diisopropylethylamine (DIPEA), pyridine, and the like. In some embodiments, it can be advantageous to use the base in excess compared to the compound of the formula I. In some embodiments, the base is used in about a 5%molar excess to about a 5-fold excess. In some embodiments, the base is used in about a 3-fold excess. In some embodiments, the inorganic base is NaHCO₃. In some embodiments, X in the acryloyl reagent is chlorine. In some embodiments, it can be advantageous to use the acryloyl reagent in excess compared to the compound of the formula I. In some embodiments, the acryloyl reagent is used in about a 5% molar excess to about a 50% molar excess. In some embodiments, the acryloyl reagent is used in about a 10% molar excess to about a 30% molar excess. In some embodiments, the acryloyl reagent is used in about a 20% molar excess.

The reaction of Step (a) can be carried out in the presence of a solvent or a solvent mixture. Exemplary solvents include, but are not limited to, methylene chloride (DCM), N,N-dimethylformamide (DMF), tetrahydrofuran (THF), ethyl acetate (EtOAc), acetone, acetonitrile (CH₃CN), dimethylsulfoxide (DMSO), and the like. In some embodiments, the solvent is aprotic. In some embodiments, the aprotic solvent is EtOAc. In some embodiments, the aprotic solvent is EtOAc, DCM, or THF. In some embodiments, the aprotic solvent can be mixed with water, where the aprotic solvent is water miscible. In some embodiments, the solvent is a mixture of THF and water. It can be advantageous to cool the reaction before or during the addition of acryloyl reagent to the reaction mixture. In some embodiments, the reaction is carried out at a temperature of between about −10° C. to about 20° C. In some embodiments, the reaction is carried out at a temperature of between about −10° C. to about 0° C.

In Step (b) of Scheme 1, the compound of the formula II is reacted with a thioacetate reagent of the formula MSAc, wherein M is H, Li, Na or K, and the like. In some embodiments, the thioacetate reagent is KSAc. The acid in Step (b) can be any acid conventionally known in the art. Examples of suitable acids include, but are not limited to, acetic acid, trifluoroacetic acid, p-toluenesulfonic acid, triflic acid, methanesulfonic acid, and the like. In some embodiments, the acid is acetic acid. In some embodiment, it can be advantageous to use the acid in excess compared to the compound of the formula II. In some embodiments, the acid is used in about a 2-fold to about a 5-fold excess. In some embodiments, the base is used in about a 2- to about 2.5-fold excess.

The reaction of step (b) can be carried out in the presence of a solvent or a mixture of a solvent and water. Exemplary solvents include, but are not limited to, methylene chloride (DCM), N,N-dimethylformamide (DMF), tetrahydrofuran (THF), ethyl acetate (EtOAc), acetone, acetonitrile (CH₃CN), dioxane, dimethylsulfoxide (DMSO), and the like. In some embodiments, the solvent is a mixture of water and a solvent. In some embodiments, the solvent is a mixture of water and dioxane. It can be advantageous to warm the reaction mixture. In some embodiments, the reaction is carried out at a temperature of between about 25° C. to about 75° C. In some embodiments, the reaction is carried out at a temperature of between about 30° C. to about 60° C. In some embodiments, the reaction is carried out at a temperature of between about 40° C. to about 60° C. In some embodiments, it can be advantageous to use the thioacetate reagent in excess compared to the compound of the formula II. In some embodiments, the thioacetate reagent is used in about a 5% molar excess to about a 50% molar excess. In some embodiments, the thioacetate reagent is used in about a 10% molar excess to about a 30% molar excess. In some embodiments, the thioacetate reagent is used in about a 10% molar excess.

In Step (c) of Scheme 1, the compound of the formula III is alkylated with an alkylating agent in the presence of a base and a solvent to provide a compound of the formula V. The alkylating agent of Step (c) can be a compound of the formula X¹—R³, wherein X¹ is a leaving group such as Cl, Br, I, triflate (—OTf), tosylate (—OTs), mesylate (—OMs), and the like, and R³ is C₁-C₆ alkyl optionally substituted with one or more halogen atoms or C₁-C₃ alkyl-C₃-C₆ cycloalkyl optionally substituted with one or more halogen atoms. In some embodiments, X¹ is iodine. Alternatively, the alkylating agent of Step (c) can be a compound of formula CH₂═CHCF₃. The base in Step (c) can be lithium hydroxide (LiOH), sodium hydroxide (NaOH), potassium hydroxide (KOH), cesium hydroxide (CsOH), calcium hydroxide (Ca(OH)₂), sodium hydride (NaH), lithium hydride (LiH), potassium hydride (KH), sodium methoxide (NaOCH₃), sodium ethoxide (NaOCH₂CH₃), and the like. In some embodiments, it can be advantageous to use the base in excess compared to the compound of the formula V. In some embodiments, the base is used in about a 2-fold to about a 5-fold excess. In some embodiments, the base is used in about a 3-fold excess. In some embodiments, the base is NaOCH₃.

The reaction of Step (c) can be carried out in the presence of a solvent or a mixture of water and a solvent. Exemplary solvents include, but are not limited to, N,N-dimethylformamide (DMF), tetrahydrofuran (THF), ethyl acetate (EtOAc), acetone, acetonitrile (CH₃CN), dioxane, dimethylsulfoxide (DMSO), methanol (MeOH), ethanol (EtOH), iso-propanol (i-PrOH), n-butanol (n-BuOH), and the like. In some embodiments, the solvent is MeOH. In some embodiments, the reaction can be carried out at room temperature. It can be advantageous to warm the reaction mixture. In some embodiments, the reaction is carried out at a temperature of between about 25° C. to about 75° C. In some embodiments, the reaction is carried out at a temperature of between about 30° C. to about 60° C. In some embodiments, the reaction is carried out at a temperature of between about 40° C. to about 60° C.

Alternatively, the processes of the present disclosure can be described according to Scheme 2.

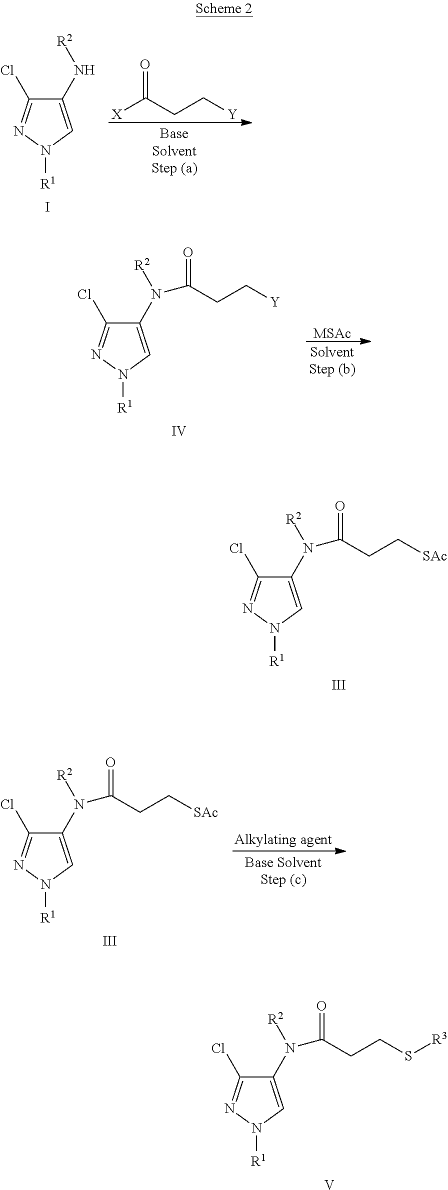

In Step (a) of Scheme 2, the compound of the formula I is acylated with an acryloyl reagent of the formula X²—C(O)CH₂CH₂Y, wherein X² is a leaving group such as F, Cl, Br, I, OC(O)C₁-C₆ alkyl, —OC(O)C₆-C₁₀ aryl. Y is a leaving group such as Cl, Br, I, triflate (—OTf), tosylate (—OTs), mesylate (—OMs), and the like, in the presence of a base. The base in Step (a) can be an inorganic base, such as sodium bicarbonate (NaHCO₃), sodium carbonate (Na₂CO₃), calcium carbonate (CaCO₃), cesium carbonate (Cs₂CO₃), lithium carbonate (Li₂CO₃), potassium carbonate (K₂CO₃), lithium hydroxide (LiOH), sodium hydroxide (NaOH), potassium hydroxide (KOH), cesium hydroxide (CsOH), calcium hydroxide (Ca(OH)₂), sodium diphosphate (Na₂HPO₄), potassium phosphate (K₃PO₄), and the like. Alternatively, the base in Step (a) can be an organic base, such as triethylamine (TEA), diisopropylethylamine (DIPEA), pyridine, and the like. In some embodiments, it can be advantageous to use the base in excess compared to the compound of the formula I. In some embodiments, the base is used in about a 5% molar excess to about a 5-fold excess. In some embodiments, the base is used in about a 2-fold excess. In some embodiments, the inorganic base is NaHCO₃. In some embodiments, X² and Y are Cl. In some embodiments, it can be advantageous to use the acryloyl reagent in excess compared to the compound of the formula I. In some embodiments, the acryloyl reagent is used in about a 5% molar excess to about a 50% molar excess. In some embodiments, the acryloyl reagent is used in about a 10% molar excess to about a 30% molar excess. In some embodiments, the acryloyl reagent is used in about a 10% molar excess.

The reaction of Step (a) can be carried out in the presence of a solvent or a mixture of a solvent and water. Exemplary solvents include, but are not limited to, methylene chloride (DCM), N,N-dimethylformamide (DMF), tetrahydrofuran (THF), ethyl acetate (EtOAc), acetone, acetonitrile (CH₃CN), dimethylsulfoxide (DMSO), and the like. In some embodiments, the solvent is EtOAc or THF. In some embodiments, the solvent can be mixed with water. In some embodiments, the solvent is a mixture of THF and water. In some embodiments, the reaction of Step (a) can be carried out at room temperature. In some embodiments, it can be advantageous to cool the reaction before or during the addition of acryloyl reagent to the reaction mixture. In some embodiments, the reaction is carried out at a temperature of between about −10° C. to about 20° C. In some embodiments, the reaction is carried out at a temperature of between about −10° C. to about 0° C. Alternatively, it can be advantageous to warm the reaction after the addition of the acryloyl reagent. In some embodiments, the reaction is carried out at a temperature of between about 20° C. to about 50° C. In some embodiments, the reaction is carried out at a temperature of between about 30° C. to about 40° C.

In Step (b) of Scheme 2, the compound of the formula IV is reacted with a thioacetate reagent of the formula MSAc, wherein M is H, Li, Na or K, and the like. In some embodiments, the thioacetate reagent is KSAc. The reaction can be carried out in the presence of a solvent, or a mixture of a solvent and water. Exemplary solvents include, but are not limited to, methylene chloride (DCM), N,N-dimethylformamide (DMF), tetrahydrofuran (THF), ethyl acetate (EtOAc), acetone, acetonitrile (CH₃CN), dioxane, dimethylsulfoxide (DMSO), and the like. In some embodiments, the solvent is a mixture of water and a solvent. In some embodiments, the solvent is acetone. It can be advantageous to warm the reaction mixture. In some embodiments, the reaction is carried out at a temperature of between about 25° C. to about 75° C. In some embodiments, the reaction is carried out at a temperature of between about 30° C. to about 60° C. In some embodiments, the reaction is carried out at a temperature of between about 40° C. to about 60° C. In some embodiments, it can be advantageous to use the thioacetate reagent in excess compared to the compound of the formula IV. In some embodiments, the thioacetate reagent is used in about a 5% molar excess to about a 50% molar excess. In some embodiments, the thioacetate reagent is used in about a 10% molar excess to about a 30% molar excess. In some embodiments, the thioacetate reagent is used in about a 10% molar excess.

In Step (c) of Scheme 2, the compound of the formula III is alkylated with an alkylating agent, in the presence of a base and a solvent to provide a compound of the formula V. The alkylating agent of Step (c) can be a compound of the formula X¹—R³, wherein X¹ is a leaving group such as Cl, Br, I, triflate (—OTf), tosylate (—OTs), mesylate (—OMs), and the like, and R³ is C₁-C₆ alkyl optionally substituted with one or more halogen atoms or C₁-C₃ alkyl-C₃-C₆ cycloalkyl optionally substituted with one or more halogen atoms. In some embodiments, X¹ is iodine. Alternatively, the alkylating agent of Step (c) can be a compound of formula CH₂═CHCF₃. The base in Step (c) can be lithium hydroxide (LiOH), sodium hydroxide (NaOH), potassium hydroxide (KOH), cesium hydroxide (CsOH), calcium hydroxide (Ca(OH)₂), sodium hydride (NaH), lithium hydride (LiH), potassium hydride (KH), sodium methoxide (NaOCH₃), sodium ethoxide (NaOCH₂CH₃), and the like. In some embodiments, it can be advantageous to use the base in excess compared to the compound of the formula III. In some embodiments, the base is used in about a 2-fold to about a 5-fold excess. In some embodiments, the base is used in about a 3-fold excess. In some embodiments, the base is NaOCH₃.

The reaction of Step (c) can be carried out in the presence of a solvent or a mixture of water and a solvent. Exemplary solvents include, but are not limited to, N,N-dimethylformamide (DMF), tetrahydrofuran (THF), ethyl acetate (EtOAc), acetone, acetonitrile (CH₃CN), dioxane, dimethylsulfoxide (DMSO), methanol (MeOH), ethanol (EtOH), iso-propanol (i-PrOH), n-butanol (n-BuOH), and the like. In some embodiments, the solvent is MeOH. In some embodiments, the solvent is a mixture of water and a solvent. In some embodiments, the reaction can be carried out at room temperature. It can be advantageous to warm the reaction mixture. In some embodiments, the reaction is carried out at a temperature of between about 25° C. to about 75° C. In some embodiments, the reaction is carried out at a temperature of between about 30° C. to about 60° C. In some embodiments, the reaction is carried out at a temperature of between about 40° C. to about 60° C.

In some embodiments, the present disclosure provides processes for the preparation of pesticidal thioethers.

In some embodiments, the present disclosure provides a process for preparing a compound of the formula V

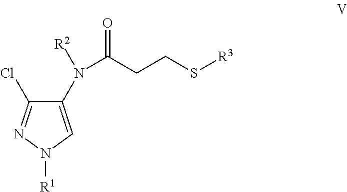

wherein R¹ is H or pyridin-3-yl; R² is H or C₁-C₆ alkyl; R³ is C₁-C₆ alkyl optionally substituted with one or more halogen atoms or C₁-C₃ alkyl-C₃-C₆ cycloalkyl optionally substituted with one or more halogen atoms,
comprising

a. contacting a compound of the formula I

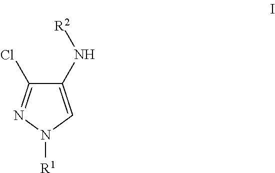

wherein R¹ is H or pyridin-3-yl; and R² is H or C₁-C₆ alkyl, with a compound of the formula X—C(O)CH═CH₂, wherein X in a leaving group, in the presence of a base and a solvent to provide a compound of the formula II

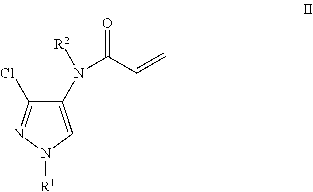

wherein R¹ is H or pyridin-3-yl; and R² is H or C₁-C₆ alkyl; or

b. contacting a compound of the formula II

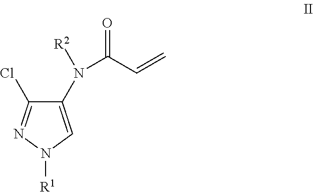

wherein R¹ is H or pyridin-3-yl; and R² is H or C₁-C₆ alkyl, with a thioacetate in the presence of an acid and a solvent to provide the compound of the formula III

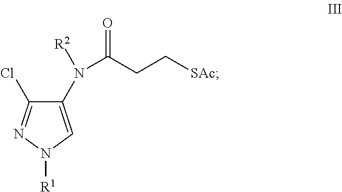

or

c. contacting a compound of the formula III

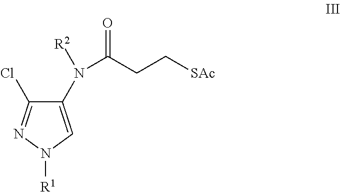

wherein R¹ is H or pyridin-3-yl; and R² is H or C₁-C₆ alkyl, with an alkylating agent in the presence of a base and a solvent to provide a compound of the formula V.

Alternatively, in some embodiments, the present disclosure provides a process for preparing a compound of the formula V

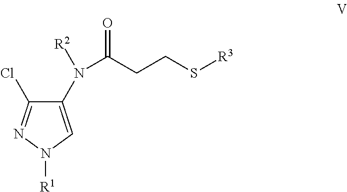

wherein R¹ is H or pyridin-3-yl; R² is H or C₁-C₆ alkyl; R³ is C₁-C₆ alkyl optionally substituted with one or more halogen atoms or C₁-C₃ alkyl-C₃-C₆ cycloalkyl optionally substituted with one or more halogen atoms,
comprising

a. contacting a compound of the formula I

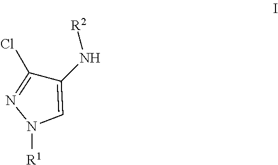

wherein R¹ is H or pyridin-3-yl; and R² is H or C₁-C₆ alkyl, with a compound of the formula X—C(O)CH₂CH₂Y, wherein X is a leaving group, in the presence of a base and a solvent to provide a compound of the formula IV

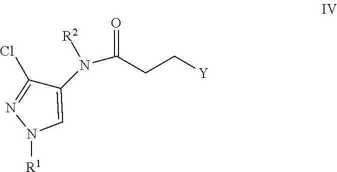

wherein R¹ is H or pyridin-3-yl; R² is H or C₁-C₆ alkyl and Y is Cl, Br, OTs or OMs; or

b. contacting a compound of the formula IV

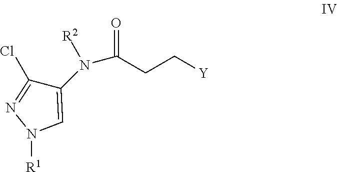

wherein R¹ is H or pyridin-3-yl; and R² is H or C₁-C₆ alkyl, with a thioacetate in the presence of a solvent to provide the compound of the formula III

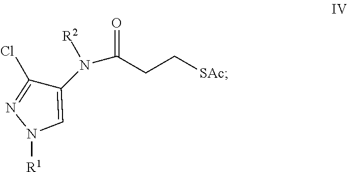

or

c. contacting a compound of the formula III

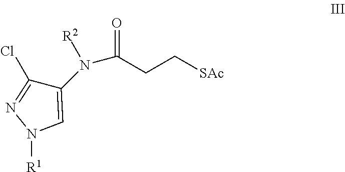

wherein R¹ is H or pyridin-3-yl; and R² is H or C₁-C₆ alkyl, with an alkylating agent in the presence of a base and a solvent to provide a compound of the formula V.

In some embodiments, the process comprises step a and step b. In some embodiments, the process comprises step a, step b, and step c. In some embodiments, the process comprises step a. In some embodiments, the process comprises step b. In some embodiments, the process comprises step c.

In some embodiments, R¹ is H. In some embodiments, R¹ is pyridin-3-yl. In some embodiments, R² is H. In some embodiments, R² is ethyl. In some embodiments, R³ is 3,3,3-trifluoropropyl. In some embodiments, R¹ is H and R² is H. In some embodiments, R¹ is pyridin-3-yl and R² is H. In some embodiments, R¹ is H and R² is ethyl. In some embodiments, R¹ is pyridin-3-yl and R² is ethyl. In some embodiments, R¹ is H, R² is H and R³ is 3,3,3-trifluoropropyl. In some embodiments, R¹ is pyridin-3-yl, R² is H and R³ is 3,3,3-trifluoropropyl. In some embodiments, R¹ is H, R² is ethyl and R³ is 3,3,3-trifluoropropyl. In some embodiments, R¹ is pyridin-3-yl, R² is ethyl and R³ is 3,3,3-trifluoropropyl.

In an alternative embodiment, the compound of the formula V can be prepared from a compound of the formula III according to a process as shown in Scheme 3.

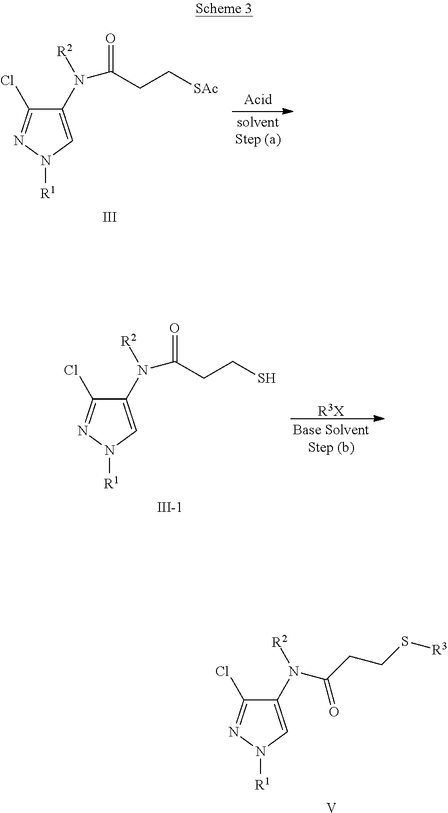

In Step (a) of the process of Scheme 3, a compound of the formula III is treated with an acid in the presence of a solvent to provide a compound of the formula III-1. Suitable acids include, but are not limited to, HCl, HBr, H₂SO₄, H₃PO₄, and the like. Exemplary solvents include, but are not limited to, N,N-dimethylformamide (DMF), tetrahydrofuran (THF), ethyl acetate (EtOAc), acetone, acetonitrile (CH₃CN), dioxane, dimethylsulfoxide (DMSO), methanol (MeOH), ethanol (EtOH), iso-propanol (i-PrOH), n-butanol (n-BuOH), and the like. In some embodiments, the solvent is MeOH. In some embodiments, the solvent is a mixture of water and a solvent. In some embodiments, it can be advantageous to cool the reaction before or during the addition of HCl to the reaction mixture. In some embodiments, the reaction is carried out at a temperature of between about −10° C. to about 20° C. In some embodiments, the reaction is carried out at a temperature of between about −10° C. to about 0° C. In some embodiments, the reaction is carried out at a temperature of about 0° C. for the addition of the acid. In some embodiments, it can be advantageous to use an excess of the acid relative to the compound of the formula III. In some embodiments, the acid is used in an excess of from about 5 to about 75-fold excess. In some embodiments, the acid is used in an excess of from about 15 to about 35-fold excess.

In Step (b) of Scheme 3, the compound of the formula III-1 is alkylated with an alkylating agent in the presence of a base and a solvent to provide a compound of the formula V. The alkylating agent of Step (b) can be a compound of the formula R³X wherein R³ is substituted or unsubstituted C₁-C₆ alkyl, or substituted or unsubstituted C₁-C₃ alkyl-C₃-C₆ cycloalkyl, and X is a leaving group such as Cl, Br, I, triflate (—OTf), tosylate (—OTs), mesylate (—OMs), and the like. The base in Step (b) can be lithium hydroxide (LiOH), sodium hydroxide (NaOH), potassium hydroxide (KOH), cesium hydroxide (CsOH), calcium hydroxide (Ca(OH)₂), sodium hydride (NaH), lithium hydride (LiH), potassium hydride (KH), sodium methoxide (NaOCH₃), sodium ethoxide (NaOCH₂CH₃), and the like. In some embodiments, it can be advantageous to use the base in excess compared to the compound of the formula III-1. In some embodiments, the base is used in about a 2-fold to about a 5-fold excess. In some embodiments, the base is used in about a 3-fold excess. In some embodiments, the base is NaOCH₃.

The reaction of Step (b) can be carried out in the presence of a solvent or a mixture of water and a solvent. Exemplary solvents include, but are not limited to, N,N-dimethylformamide (DMF), tetrahydrofuran (THF), ethyl acetate (EtOAc), acetone, acetonitrile (CH₃CN), dioxane, dimethylsulfoxide (DMSO), nitromethane, methanol (MeOH), ethanol (EtOH), iso-propanol (i-PrOH), n-butanol (n-BuOH), and the like. In some embodiments, the solvent is MeOH. In some embodiments, the solvent is a mixture of water and a solvent. In some embodiments, the reaction can be carried out at room temperature. It can be advantageous to warm the reaction mixture. In some embodiments, the reaction is carried out at a temperature of between about 25° C. to about 75° C. In some embodiments, the reaction is carried out at a temperature of between about 30° C. to about 60° C. In some embodiments, the reaction is carried out at a temperature of between about 40° C. to about 60° C.

In an alternative embodiment, the compound of the formula Vd can be prepared from a compound of the formula IIId according to a process as shown in Scheme 4.

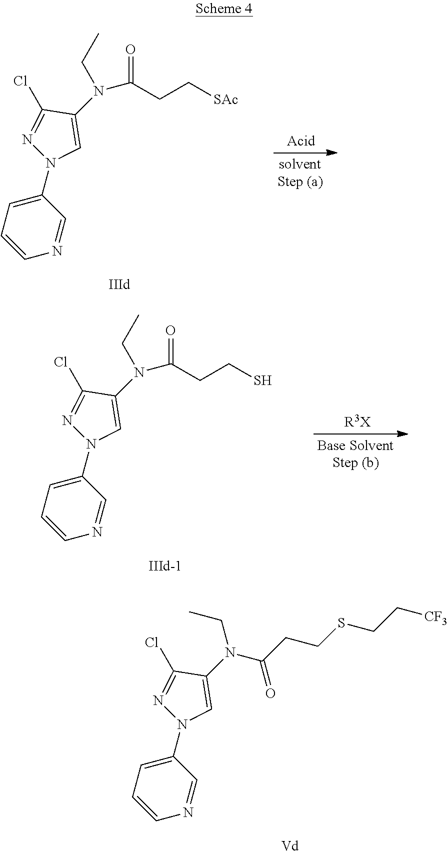

In the process of Scheme 4, a compound of the formula IIId is treated with an acid in the presence of a solvent to provide a compound of the formula IIId-1. Suitable acids include, but are not limited to, HCl, HBr, H₂SO₄, H₃PO₄, and the like. Exemplary solvents include, but are not limited to, N,N-dimethylformamide (DMF), tetrahydrofuran (THF), ethyl acetate (EtOAc), acetone, acetonitrile (CH₃CN), dioxane, dimethylsulfoxide (DMSO), methanol (MeOH), ethanol (EtOH), iso-propanol (i-PrOH), n-butanol (n-BuOH), and the like. In some embodiments, the solvent is MeOH. In some embodiments, the solvent is a mixture of water and a solvent. In some embodiments, it can be advantageous to cool the reaction before or during the addition of HCl to the reaction mixture. In some embodiments, the reaction is carried out at a temperature of between about −10° C. to about 20° C. In some embodiments, the reaction is carried out at a temperature of between about −10° C. to about 0° C. In some embodiments, the reaction is carried out at a temperature of about 0° C. for the addition of the acid. In some embodiments, it can be advantageous to use an excess of the acid relative to the compound of the formula IIId. In some embodiments, the acid is used in an excess of from about 5 to about 75-fold excess. In some embodiments, the acid is used in an excess of from about 15 to about 35-fold excess.

In Step (b) of Scheme 4, the compound of the formula IIId-1 is alkylated with an alkylating agent, in the presence of a base and a solvent to provide a compound of the formula Vd. The alkylating agent of Step (b) can be a compound of the formula R³X wherein R³ is substituted or unsubstituted C₁-C₆ alkyl, or substituted or unsubstituted C₁-C₃ alkyl-C₃-C₆ cycloalkyl, and X is a leaving group such as Cl, Br, I, triflate (—OTf), tosylate (—OTs), mesylate (—OMs), and the like. The base in Step (b) can be lithium hydroxide (LiOH), sodium hydroxide (NaOH), potassium hydroxide (KOH), cesium hydroxide (CsOH), calcium hydroxide (Ca(OH)₂), sodium hydride (NaH), lithium hydride (LiH), potassium hydride (KH), sodium methoxide (NaOCH₃), sodium ethoxide (NaOCH₂CH₃), and the like. In some embodiments, it can be advantageous to use the inorganic base in excess compared to the compound of the formula IIId-1. In some embodiments, the base is used in about a 2-fold to about a 5-fold excess. In some embodiments, the base is used in about a 3-fold excess. In some embodiments, the base is NaOCH₃.

The reaction of Step (b) can be carried out in the presence of a solvent or a mixture of water and a solvent. Exemplary solvents include, but are not limited to, N,N-dimethylformamide (DMF), tetrahydrofuran (THF), ethyl acetate (EtOAc), acetone, acetonitrile (CH₃CN), dioxane, dimethylsulfoxide (DMSO), methanol (MeOH), ethanol (EtOH), iso-propanol (i-PrOH), n-butanol (n-BuOH), and the like. In some embodiments, the solvent is MeOH. In some embodiments, the solvent is a mixture of water and a solvent. In some embodiments, the reaction can be carried out at room temperature. It can be advantageous to warm the reaction mixture. In some embodiments, the reaction is carried out at a temperature of between about 25° C. to about 75° C. In some embodiments, the reaction is carried out at a temperature of between about 30° C. to about 60° C. In some embodiments, the reaction is carried out at a temperature of between about 40° C. to about 60° C.

### EXAMPLES

### Materials and Methods

These examples are for illustration purposes and are not to be construed as limiting this disclosure to only the embodiments disclosed in these examples. Starting materials, reagents, and solvents that were obtained from commercial sources were used without further purification. Melting points are uncorrected. Examples using “room temperature” were conducted in climate controlled laboratories with temperatures ranging from about 20° C. to about 24° C. Molecules are given their known names, named according to naming programs within Accelrys Draw, ChemDraw, or ACD Name Pro. If such programs are unable to name a molecule, such molecule is named using conventional naming rules. ¹H NMR spectral data are in ppm (δ) and were recorded at 300, 400, 500, or 600 MHz; ¹³C NMR spectral data are in ppm (δ) and were recorded at 75, 100, or 150 MHz, and ¹⁹F NMR spectral data are in ppm (δ) and were recorded at 376 MHz, unless otherwise stated.

3-Chloro-1H-pyrazol-4-amine hydrochloride, compound Ia, was prepared according to the method described in U.S. Pat. No. 9,102,655, incorporated herein by reference for the preparation of compound Ia, referred to therein as compound 1a. 3-Chloro-N-ethyl-1H-pyrazol-4-amine, compound Ib, was prepared was prepared according to the method described in U.S. Pat. No. 9,029,554, incorporated herein by reference for the preparation of compound Ib, referred to therein as compound 7a. 3-(3-Chloro-4-amino-1H-pyrazol-1-yl)pyridine, compound Ic was prepared was prepared according to the method described in U.S. Pat. No. 9,414,594, incorporated herein by reference for the preparation of compound Ic, referred to therein as compound 5d. 3-Chloro-N-ethyl-1-(pyridin-3-yl)-1H-pyrazol-amine, compound Id was prepared was prepared according to the method described in U.S. Pat. No. 9,102,655, incorporated herein by reference for the preparation of compound Id, referred to therein as compound 1 d.

### Chemistry Examples

### Example 1

### Preparation of N-(3-chloro-1H-pyrazol-4-yl)acrylamide (IIa)

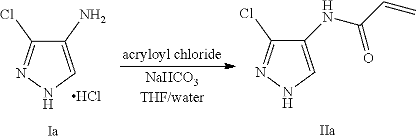

A 4-neck, 500-mL round bottom flask was charged with 3-chloro-1H-pyrazol-4-amine.HCl (15 g, 128 mmol), THF (50 mL), and water (50 mL) Sodium bicarbonate (32.2 g, 383 mmol) was added in portions to control off-gassing, and the mixture was cooled to 5° C. Acryloyl chloride (12.44 mL, 153 mmol) was added at <20° C. and the reaction was stirred for 2 h, after which the reaction was diluted with water (100 mL) and EtOAc (100 mL) The organic layer was concentrated to dryness to afford a white solid, which was suspended in MTBE (50 mL) and stirred for 2 h. The suspension was filtered and the solid was rinsed with MTBE (50 mL) to afford the desired product, N-(3-chloro-1H-pyrazol-4-yl)acrylamide (IIa), as a white solid after drying (14.8 g, 68% yield), mp: 182° C. (decomposition). ¹H NMR (400 MHz, DMSO-d₆) δ 12.96 (s, 1H), 9.77 (s, 1H), 8.10 (s. 1H), 6.58 (dd, J=17.0, 10.2 Hz, 1H), 6.23 (dd, J=17.0, 2.1 Hz, 1H), 5.73 (dd, 10.2, 2.1 Hz, 1H). ¹³C NMR (101 MHz, DMSO-d₆) δ 162.69, 130.76, 130.14, 126.62, 123.60, 116.53. ESIMS: m/z 172.0 ([M+H]⁺).

### Example 2

### Preparation of S-(3-((3-chloro-1H-pyrazol-4-yl)amino)-3-oxopropyl) ethanethioate (IIIa)

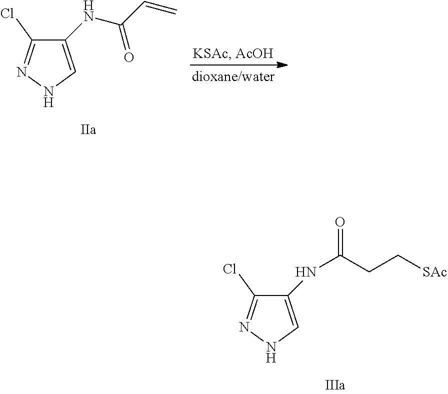

A 100-mL round bottom flask equipped with a magnetic stirrer and a temperature probe was charged with potassium thioacetate (5.32 g, 23 3 mmol), water (8 mL), and dioxane (20 mL) Acetic acid (2.8 mL, 48.9 mmol) was added and the solution was stirred for 15 min. N-(3-Chloro-1H-pyrazol-4-yl)acrylamide (4.0 g, 23 3 mmol) was added and the reaction mixture was heated at 50° C. for 5 h, at which time HPLC analysis indicated complete conversion of N-(3-chloro-1H-pyrazol-4-yl)acrylamide to the product. The solution was cooled to room temperature and transferred to a separatory funnel Saturated aq. NaHCO₃ solution (25 mL) and EtOAc (150 mL) were added. The layers were separated, and the aqueous phase was extracted with EtOAc (50 mL). The organic layers were washed with brine (50 mL), dried over anhydrous Na₂SO₄, and concentrated under reduced pressure to afford an off-white solid. The crude product was suspended in 1:1 MTBE/hexanes (40 mL) and stirred for 1 h. The solid was filtered and washed with hexanes (20 mL) to afford the desired product, S-(3-((3-chloro-1H-pyrazol-4-yl)amino)-3-oxopropyl) ethanethioate (Ma), as a white solid (4.94 g, 86% yield, 96% HPLC purity). mp 140-143° C. ¹H NMR (400 MHz, DMSO-d₆): 2.89 (s, 1H), 9.58 (s, 1H), 8.00 (s, 1H), 3.05 (t, J=6.9 Hz, 2H), 2.64 (t, J=6.9 Hz, 2H), 2.32 (s, 3H). ¹³C NMR (101 MHz, DMSO-d₆): 195.3, 168.8, 130.2, 123.6, 116.5, 34.7, 30.5, 24.3. ESIMS m/z 247.8 ([M+H]⁺).

### Example 3

### Preparation of N-(3-chloro-1H-pyrazol-4-yl)-3-((3,3,3-trifluoropropyl)thio)propionamide (Va)

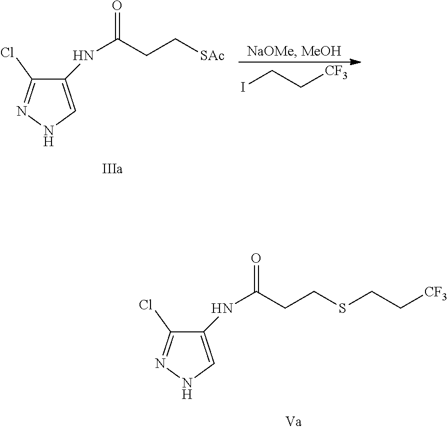

A 50-mL round bottom flask was charged with S-(3-((3-chloro-1H-pyrazol-4-yl)amino)-3-oxopropyl) ethanethioate (1.1 g, 4.44 mmol) and methanol (22 mL) and the mixture was stirred under a flow of nitrogen for 15 min. Sodium methoxide (0.725 g, 13 4 mmol) was added and the suspension was stirred under nitrogen for 5 min. 1,1,1-Trifluoro-3-iodopropane (1.56 mL, 13.3 mmol) was added and the reaction was heated at 50° C. for 4 h, at which time HPLC analysis revealed complete conversion of S-(3-((3-chloro-1H-pyrazol-4-yl)amino)-3-oxopropyl) ethanethioate to the product. The reaction was cooled to room temperature and transferred to a separatory funnel. EtOAc (100 mL) and water (50 mL) were added, and the layers were separated. The aqueous phase was extracted with EtOAc (50 mL) The combined organic layers were washed with brine (25 mL), dried over anhydrous Na₂So₄, and concentrated under reduced pressure. The residue was purified by flash column chromatography (20-80% EtOAc/hexanes) to afford an oil which solidified over 12 h to give the desired product as a white solid (1.11 g, 83% yield, 97% HPLC purity). mp 84-85° C. ¹H NMR (400 MHz, DMSO-d₆): 12.88 (s, 1H), 9.57 (s, 1H), 8.00 (s, 1H), 2.81 (t, J=7.0 Hz, 2H), 2.75-2.68 (m, 2H), 2.65 (t, J=7.0 Hz, 2H), 2.61-2.52 (m, 2H). ¹³C NMR (100 MHz, DMSO-d₆): 169.03, 130.03, 126.60 (q, J=277.4 Hz), 123.50, 116.65, 35.29, 33.48 (q, J=27.2 Hz), 26.90, 23.10. ESIMS m/z 301.8 ([M+H]⁺).

### Example 4

### Preparation of N-(3-chloro-1H-pyrazol-4-yl)-N-ethylacrylamide (IIb)

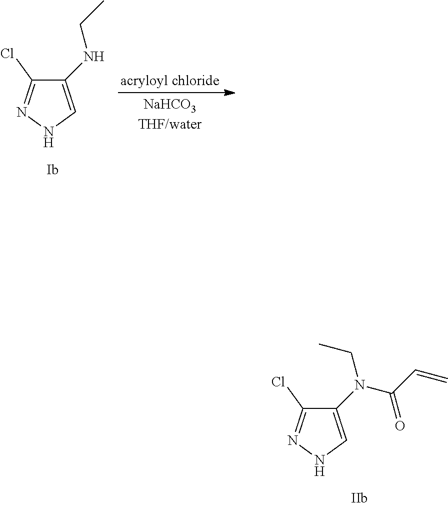

A 4-neck, 100-mL round bottom flask was charged with 3-chloro-N-ethyl-1H-pyrazol-4-amine (2.5 g, 17.17 mmol), THF (10 mL), and water (10 mL). Sodium bicarbonate (3.46 g, 41.2 mmol) was added in portions, and the mixture was cooled to 5° C. Acryloyl chloride (1.34 mL, 16.48 mmol) was added at <20° C. and the reaction was stirred for 2 h, after which it was diluted with water (20 mL) and EtOAc (20 mL) The organic layer was concentrated to dryness to afford a white solid, which was suspended in MTBE (20 mL) and stirred for 2 h. It was filtered and the solid was rinsed with MTBE (10 mL) to afford the desired product N-(3-chloro-1H-pyrazol-4-yl)-N-ethylacrylamide (IIb) as a white solid after drying (2.4 g, 70% yield). mp: 156-160° C. ¹H NMR (400 MHz, DMSO-d₆) δ 8.05 (s, 1H), 6.17 (dd, J=16.8, 2.6 Hz, 1H), 6.06 (dd, J=16.8, 10.0 Hz, 1H), 5.60 (dd, J=10.0, 2.6 Hz, 1H), 3.58 (q, J=7.1 Hz, 2H), 1.03 (t, J=7.2 Hz, 3H). ¹³C NMR (101 MHz, DMSO-d₆) δ 164.82, 136.17, 129.40, 128.02, 127.75, 119.27, 43.29, 12.65. ESIMS: m/z 200.0 ([M+H]⁺).

### Example 5

### Preparation of S-(3-((3-chloro-1H-pyrazol-4-yl)(ethyl)amino)-3-oxopropyl) ethanethioate (IIIb)

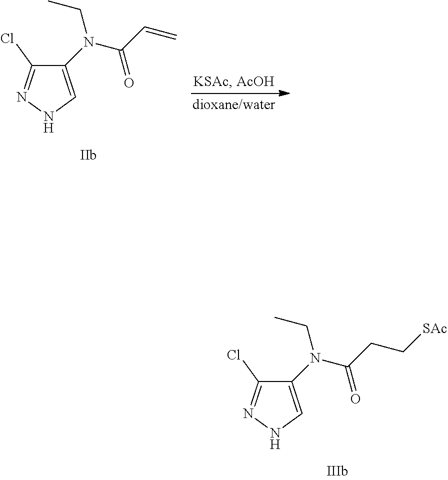

A 100-mL round bottom flask equipped with a magnetic stirrer and a temperature probe was charged with potassium thioacetate (0.63 g, 5.5 mmol), water (4 mL), and dioxane (8 mL) Acetic acid (0.66 mL, 11.5 mmol) was added and the solution was stirred for 15 min. N-(3-Chloro-1H-pyrazol-4-yl)-N-ethylacrylamide (1.1 g, 5.51 mmol) was added, and the reaction mixture was heated at 50° C. for 4 h, at which point HPLC analysis indicated complete conversion of N-(3-chloro-1H-pyrazol-4-yl)-N-ethylacrylamide to the product. The solution was cooled to room temperature and transferred to a separatory funnel Saturated NaHCO₃ solution (10 mL), water (25 mL), and EtOAc (100 mL) were added. The organic layer was separated, and the aqueous phase was extracted with EtOAc (50 mL). The combined organic layers were washed with brine (50 mL), dried over anhydrous Na₂SO₄, and concentrated under reduced pressure to afford the desired product, S-(3-((3-chloro-1H-pyrazol-4-yl)(ethyl)amino)-3-oxopropyl) ethanethioate (IIIb), as a white solid (1.3 g, 85% yield, 95% HPLC purity). mp 98-99° C. ¹H NMR (400 MHz, CDCl₃): 11.91 (s, 1H), 7.60 (s, 1H), 3.66 (q, J=6.6 Hz, 2H), 3.09 (t, J=6.8 Hz, 2H), 2.40 (t, J=6.7 Hz, 2H), 2.28 (s, 3H), 1.11 (t, J=7.1 Hz, 3H). ¹³C NMR (100 MHz, CDCl₃): 196.5, 171.8, 138.1, 128.6, 120.8, 44.1, 34.3, 30.7, 24.7, 13.1. ESIMS m/z 275.82 ([M+H]⁺).

### Example 6

### Preparation of N-(3-chloro-1H-pyrazol-4-yl)-N-ethyl-3-((3,3,3-trifluoropropyl)-thio)propanamide (Vb)

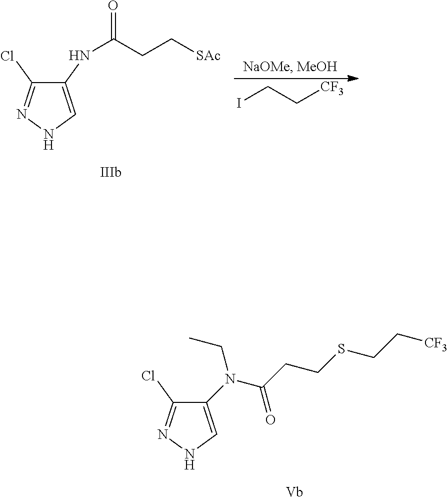

A 50-mL round bottom flask was charged with S-(3-((3-chloro-1H-pyrazol-4-yl)(ethyl)amino)-3-oxopropyl) ethanethioate (0.75 g, 2.72 mmol) and methanol (20 mL), and purged with a flow of nitrogen for 15 min. Sodium methoxide (0.44 g, 8.16 mmol) was added and the suspension was stirred under nitrogen for 5 min 1,1,1-Trifluoro-3-iodopropane (0.95 mL, 8.16 mmol) was added and the reaction was heated at 50° C. for 4 h, at which time HPLC analysis revealed complete conversion of S-(3-((3-chloro-1H-pyrazol-4-yl)(ethyl)amino)-3-oxopropyl) ethanethioate to the product. The reaction was cooled to room temperature and transferred to a separatory funnel, and EtOAc (100 mL) and water (50 mL) were added. The layers were separated and the aqueous phase was extracted with EtOAc (50 mL). The organic layers were combined and washed with brine (25 mL), dried over anhydrous Na₂SO₄, and concentrated under reduced pressure. The residue was purified by flash column chromatography (20-80% EtOAc/hexanes) to afford the desired product, N-(3-chloro-1H-pyrazol-4-yl)-N-ethyl-3-((3,3,3-trifluoropropyl)-thio)propanamide (Vb), as an oil (0.832 g, 75% yield, 91% HPLC purity). ¹H NMR (400 MHz, CDCl₃): 12.03 (s, 1H), 7.60 (s,1H), 3.67 (q, J=6.9 Hz, 2H), 2.81 (t, J=7.3 Hz, 2H), 2.65-2.61 (m, 2H), 2.49-2.21 (m, 4H), 1.11 (t, J=7.1 Hz, 3H). ESI-MS m/z 329.8 ([M+H]⁺).

### Example 8

### Preparation of N-(3-chloro-1-(pyridin-3-yl)-1H-pyrazol-4-yl)acrylamide (IIc)

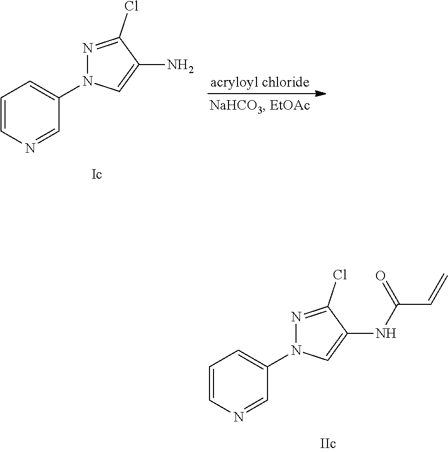 [SMILES: Nc1cn(-c2cccnc2)nc1Cl ; C=CC(=O)Nc1cn(-c2cccnc2)nc1Cl]

A 4-neck, 500-mL round bottom flask was charged with 3-chloro-1-(pyridin-3-yl)-1H-pyrazol-4-amine (14.0 g, 71.9 mmol), and DCM (200 mL). Sodium bicarbonate (18.13 g, 216 mmol) was added, and the suspension was cooled to 0° C. Acryloyl chloride (7.01 mL, 86 mmol) was added at <20° C. and the reaction was stirred for 2 h, at which point HPLC analysis indicated that the reaction was complete. The reaction was quenched with water (100 mL) The suspension was filtered and the filter cake was rinsed with water (2×50 mL). The filter cake was suspended in IPA (200 mL) and stirred at 20° C. for 1 h. Water (200 mL) was added and the resulting suspension was stirred for 2 h. The suspension was filtered and the solid was rinsed with water (2×50 mL) to afford the desired product, N-(3-chloro-1-(pyridin-3-yl)-1H-pyrazol-4-yl)acrylamide (IIc) as a white solid after drying (16.8 g, 92% yield). mp: 148-153° C. ¹H NMR (400 MHz, DMSO-d₆) δ 10.10 (s, 1H), 9.06 (d, J=2.7 Hz, 1H), 8.94 (s, 1H), 8.55 (dd,=4.7, 1.4 Hz, 1H), 8.22 (ddd, J=8.4, 2.8, 1.4 Hz, 1H), 7.55 (dd, J=8.4,4.7 Hz, 1H), 6.64 (dd, J=17.0, 10.2 Hz, 1H), 6.30 (dd, 17.1,2.0 Hz, 1H), 5.80 (dd, J=10.2, 2.0 Hz, 1H). ¹³C NMR (101 MHz, DMSO-d₆) δ 162.95, 147.56, 139.50, 135.46, 133.66, 130.39, 127.49, 125.56, 124.23, 122.56, 119.91. ESIMS: m/z 249.1 ([M+H]⁺).

### Example 9

### Preparation of S-(3-((3-chloro-1-(pyridin-3-yl)-1H-pyrazol-4-yl)amino)-3-oxopropyl) ethanethioate (IIIc)

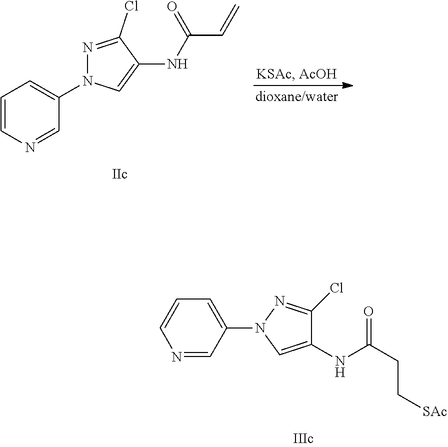

A 250-mL round bottom flask equipped with a magnetic stir bar and a temperature probe was charged with potassium thioacetate (1.837 g, 16 0 mmol), water (23 mL), and acetic acid (1.93 g, 32 mmol). The solution was stirred at room temperature for 30 min, and a solution of N-(3-chloro-1-(pyridin-3-yl)-1H-pyrazol-4-yl)acrylamide (2.0 g, 8 0 mmol) in THF (32 mL) was added. The reaction was stirred at room temperature for 14 h, at which point HPLC analysis indicated that less than 0.5% of N-(3-chloro-1-(pyridin-3-yl)-1H-pyrazol-4-yl)acrylamide remained The precipitate was collected by filtration and the solid was rinsed with EtOAc to afford 0.65 g of the desired product (97% HPLC purity). The filtrate was diluted with water (30 mL) and EtOAc (50 mL) The layers were separated and the aqueous layer was extracted with EtOAc (3×25 mL). The combined organics were washed with brine (50 mL), dried over anhydrous Na₂SO₄, and concentrated to afford 1.7 g of a crude product (88% HPLC purity). The crude product was triturated with MeOH/THF (9:1) and filtered to afford 1.3 g of the desired product as an off white solid (95.5% HPLC purity). The combined yield was 75% (1.95 g, 96% HPLC purity). mp 163-166° C. ¹H NMR (400MHz, DMSO-d₆): 9.92 (s, 1H), 9.04 (s, 1H), 8.85 (s, 1H), 8.53 (d, J=4.4 Hz, 1H), 8.20 (d, J=8.3 Hz, 1H), 7.53 (dd, J=8.2, 4.7 Hz, 1H), 3.09 (t, J=6.7 Hz, 2H), 2.72 (t, J=6.7 Hz, 2H), 2.33 (s, 3H). ¹³C NMR (101MHz, DMSO-d₆): 195.3, 169.1, 147.5, 139.4, 135.5, 133.5, 125.5, 124.2, 122.4, 119.9, 34.7, 30.5, 24.2. ESIMS m/z 324.9 ([M+H]⁺).

### Example 10

### Preparation of N-(3-chloro-1-(pyridin-3-yl)-1H-pyrazol-4-yl)-3-((3,3,3-trifluoropropyl)thio)propanamide (Vc)

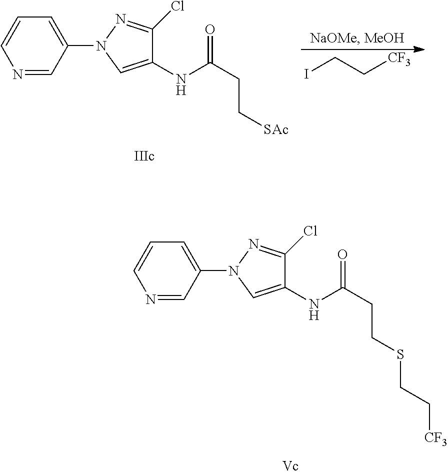

To a 50-mL round bottom flask equipped with a magnetic stir bar, a temperature probe, and a reflux condenser was charged S-(3-((3-chloro-1-(pyridin-3-yl)-1H-pyrazol-4-yl)amino)-3-oxopropyl) ethanethioate (1.0 g, 3 mmol, 96% HPLC purity) and MeOH (30 mL). To the suspension was added NaOMe (0.497 g, 9 0 mmol) and the reaction mixture was stirred at room temperature for 30 min, at which point HPLC analysis indicated that <0.3% of S-(3-((3-chloro-1-(pyridin-3-yl)-1H-pyrazol-4-yl)amino)-3-oxopropyl) ethanethioate remained. 1,1,1-Trifluoro-3-iodopropane (2.06 g, 9. 0 mmol) was added and the mixture was heated at 50° C. for 30 min, at which point HPLC analysis indicated that <1.5% of thiol intermediate remained. The reaction mixture was cooled to room temperature, filtered, and the filter cake washed with MeOH (10 mL) The filtrate was concentrated to afford crude product as an off-white solid (2.0 g, 90% HPLC purity), which was purified by flash column chromatography (0-100% EtOAc/hexanes) to afford the desired product as a white solid (1.0 g, 86%, 98.2% HPLC purity). mp 111-114° C. ¹H NMR (400 MHz, CDCl₃): 8.97 (s, 1H), 8.63 (s, 1H), 8.54 (d, J=4.6 Hz, 1H), 7.97 (d, J=9.4 Hz, 1H), 7.64 (s, 1H), 7.39 (dd, J=8.3, 4.8 Hz, 1H), 2.95 (t, J=6.8 Hz, 2H), 2.75 (m, 4H), 2.32-2.50 (m, 2H). ¹³C NMR (101 MHz, CDCl₃): 168.3, 147.9, 140.1, 136.1, 132.5, 127.4, 125.9, 124.7, 124.1, 120.0, 36.5, 34.7 (q, J=29 Hz), 27.6, 24.6. ESIMS m/z 378.9 ([M+H]⁺).

### Example 11

### Preparation of N-(3-chloro-1-(pyridin-3-yl)-1H-pyrazol-4-yl)-N-ethylacrylamide (IId)

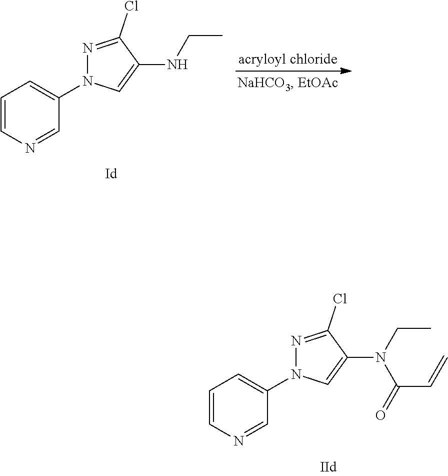 [SMILES: CCNc1cn(-c2cccnc2)nc1Cl ; C=CC(=O)N(CC)c1cn(-c2cccnc2)nc1Cl]

A 4-neck, 500-mL round bottom flask was charged with 3-chloro-N-ethyl-1-(pyridin-3-yl)-1H-pyrazol-4-amine (20.0 g, 90 mmol), and DCM (200 mL) NaHCO₃ (18.86 g, 225 mmol) was added, and the reaction was cooled to <5° C. Acryloyl chloride (8.76 mL, 108 mmol) was added dropwise at <10° C. The reaction was stirred at 20° C. for 2 h, at which point HPLC analysis indicated that the reaction was complete. The reaction was diluted with water (200 mL) (off-gassing) and the layers were separated. The aqueous layer was extracted with DCM (100 mL) and the combined organic layers were concentrated to dryness to afford a light brown oil, which was purified by column chromatography (330 g silica, 0-50% EtOAc/hexanes over 5 column volumes, hold at 50% for 5 column volumes). The fractions containing the pure product were combined and concentrated to dryness to afford N-(3-chloro-1-(pyridin-3-yl)-1H-pyrazol-4-yl)-N-ethylacrylamide (IId) as a white solid after drying under vacuum at 20° C. for 2 days (15.8 g, 64%). mp: 81-82° C. ¹H NMR (400 MHz, CDCl₃) δ 8.97 (d, J=2.7 Hz, 1H), 8.71-8.53 (m, 1H), 8.06 (ddd, J=8.3, 2.8,1.5 Hz, 1H), 7.98 (s, 1H), 7.46 (dd, J=8.3,4.7 Hz, 1H), 6.43 (dd, 16.7, 1.9 Hz, 1H), 6.18 (dd, J=16.8, 10.3 Hz, 1H), 5.75-5.50 (m, 1H), 3.78 (q, J=7.2 Hz, 2H), 1.20 (t, J=7.1 Hz, 3H). ¹³C NMR (101 MHz, CDCl₃) δ 165.77, 148.59, 141.12, 139.99, 135.65, 128.92, 127.58, 126.39, 126.22, 124.07, 123.79, 44.06, 13.02. ESIMS: m/z 277.1 ([M+H]⁺).

### Example 12

### Alternative preparation of N-(3-chloro-1-(pyridin-3-yl)-1H-pyrazol-4-yl)-N-ethylacrylamide (IId)

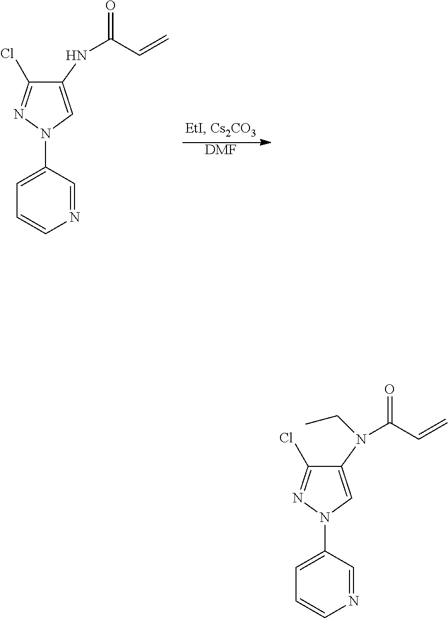

A 25-mL round bottom flask was charged with N-(3-chloro-1-(pyridin-3-yl)-1H-pyrazol-4-yl)acrylamide (0.5 g, 2.0 mmol), DMF (4 mL), and Cs₂CO₃ (1.5 g, 4.6 mmol) under nitrogen.

To the suspension was added EtI (0.2 mL, 2 5 mmol) and the reaction was stirred at room temperature for 12 h. The reaction mixture was transferred to a separatory funnel containing water (25 mL) and extracted with EtOAc (3×25 mL). The organic layers were combined and washed with water (10 mL), brine (25 mL), dried over anhydrous Na₂SO₄, and concentrated under reduced pressure. The residue was purified by flash column chromatography (10-100% EtOAc/hexanes) to afford the desired product as a pale-yellow solid (0.39 g, 70% yield, 98% HPLC purity). mp 79-82° C. ¹H NMR (400 MHz, CDCl₃): 8.94 (s, 1H), 8.61 (s, 1H), 8.04 (d, J=9.4 Hz, 1H), 7.96 (s, 1H), 7.44 (dd, J=8.0, 4.9 Hz, 1H), 6.41 (d, J=16.7 Hz, 1H), 6.16 (dd, J=16.6, 10.3 Hz, 1H), 5.61 (d, J=10.3 Hz, 1H), 3.76 (q, J=7.0 Hz, 2H), 1.18 (t, J=7.1 Hz, 3H). ¹³C NMR (100 MHz, CDCl₃): 165.9, 148.7, 141.2, 140.1, 135.8, 129.1, 127.7, 126.5, 126.3, 124.2, 123.9, 44.2, 13.1. ESI-MS m/z 277.0 ([M+H]⁺).

### Example 13

### Alternative Preparation of N-(3-chloro-1-(pyridin-3-yl)-1H-pyrazol-4-yl)-N-ethylacrylamide (IId)

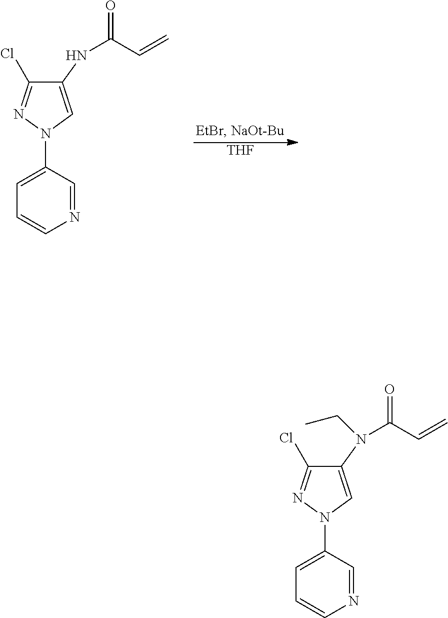 [SMILES: C=CC(=O)Nc1cn(-c2cccnc2)nc1Cl ; C=CC(=O)N(CC)c1cn(-c2cccnc2)nc1Cl ; "THF"]

Sodium tert-butoxide (0.966 g, 10 mmol) was added to a solution of N-(3-chloro-1-(pyridin-3-yl)-1H-pyrazol-4-yl)acrylamide (2.0 g, 8 mmol) in anhydrous THF (20 mL), followed by bromoethane (1.31 g, 0.9 mL, 12 mmol). The reaction mixture was heated to 58° C. and stirred at 58° C. for 22 h, at which time HPLC analysis indicated that <3% of N-(3-chloro-1-(pyridin-3-yl)-1H-pyrazol-4-yl)acrylamide remained. The reaction mixture was cooled down to room temperature, and concentrated under reduced pressure to obtain a brown residue, which was dissolved in EtOAc (50 mL) and water (35 mL) Then organic layer was separated and the aqueous layer was extracted with EtOAc (3×25 mL). The combined organic layers were washed with brine (50 mL), dried over anhydrous Na₂SO₄, filtered and concentrated to give the desired product (2.0 g) as a crude reddish oil (2.0 g). The crude oil was purified by column chromatography (0-100% EtOAc/hexanes) to afford the desired product as a sticky wax (0.782 g, 35% yield, 95.5% purity). ¹H NMR (400 MHz, DMSO-d₆): 9.08 (d, J=2.3 Hz, 1H), 8.97 (s, 1H), 8.59 (d, J=8.4 Hz, 1H), 8.23 (d, J=9.4 Hz, 1H), 7.68-7.54 (dd, J=8.7, 4.4 Hz, 1H), 6.23 (d, J=6.8 Hz, 2H), 5.74-5.57 (m, 1H), 3.65 (q, J=6.6 Hz, 2H), 1.10 (t, J=7.1 Hz, 3H). ¹³C NMR (101 MHz, CDCl₃): 165.9, 148.7, 140.1, 135.7, 129.1, 127.7, 126.5, 126.4, 124.2, 123.9, 44.2, 27.6, 13.1. ESIMS 277.0 ([M+H]⁺).

### Example 14

### Alternative preparation of N-(3-chloro-1-(pyridin-3-yl)-1H-pyrazol-4-yl)-N-ethylacrylamide (IId)

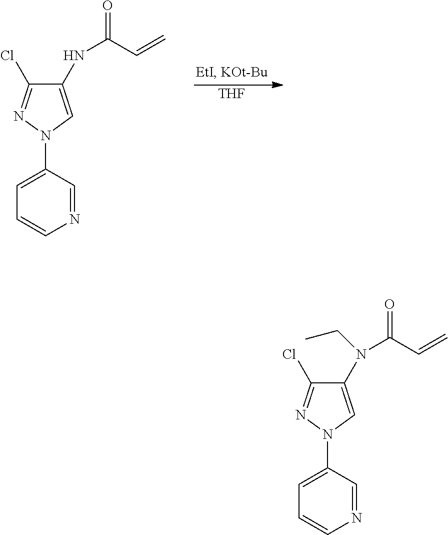 [SMILES: C=CC(=O)Nc1cn(-c2cccnc2)nc1Cl ; C=CC(=O)N(CC)c1cn(-c2cccnc2)nc1Cl ; "THF"]

Potassium tort-butoxide (0.338 g, 3.01 mmol) was added to a solution of N-(3-chloro-1-(pyridin-3-yl)-1H-pyrazol-4-yl)acrylamide (0.5 g, 2.01 mmol) in anhydrous THF (5 mL), followed by iodoethane (0.376 g, 2.41 mmol). The reaction mixture was heated to 58° C. and stirred at 58° C. for 16 h, at which time HPLC analysis indicated that <3% of N-(3-chloro-1-(pyridin-3-yl)-1H-pyrazol-4-yl)acrylamide remained. The reaction mixture was cooled to room temperature, filtered, and the filtrate was concentrated to afford a yellowish oil. The crude oil was purified by column chromatography (0-100% EtOAc/hexanes) to afford the desired product as an off-white solid (0.29 g, 52.5% yield, 97.2% purity). Analytical data was consistent with that of previously obtained samples.

### Example 15

### Preparation of S-(3-((3-chloro-1-(pyridin-3-yl)-1H-pyrazol-4-yl)(ethyl)amino)-3-oxopropyl) ethanethioate (IIId)

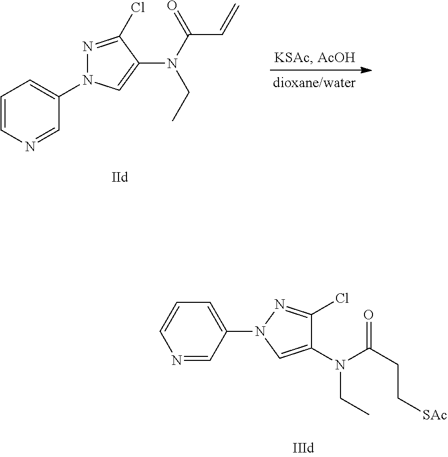

A 50-mL round bottom flask equipped with a magnetic stirrer and a temperature probe was charged with potassium thioacetate (1.24 g, 10.84 mmol), water (3 mL), and dioxane (8 mL) Acetic acid (0.65 mL, 11.3 mmol) was added and the solution was stirred for 15 min N-(3-Chloro-1-(pyridin-3-yl)-1H-pyrazol-4-yl)-N-ethylacrylamide (1.5 g, 5.42 mmol) was added, and the reaction mixture was heated at 50° C. for 5 h, at which time HPLC analysis indicated complete conversion of N-(3-chloro-1-(pyridin-3-yl)-1H-pyrazol-4-yl)-N-ethylacrylamide to the product. The solution was cooled to room temperature, transferred to a separatory funnel and water (50 mL), and EtOAc (100 mL) were added. The layers were separated, and the aqueous phase was extracted with EtOAc (25 mL) The combined organic layers were washed with brine (25 mL), dried over anhydrous Na₂SO₄, and concentrated under reduced pressure. The residue was purified by flash column chromatography (50-100% EtOAc/hexanes) to afford the desired product as an oil (1.7 g, 89% yield, 98% HPLC purity). ¹H NMR (400 MHz, CDCl₃): 8.95 (s, 1H), 8.61 (d, J=4.6 Hz, 1H), 8.05 (d, J=8.3 Hz, 1H), 7.99-7.88 (m, 1H), 7.45 (m, 1H), 3.70 (q, J=6.9 Hz, 2H), 3.10 (t, J=6.9 Hz, 2H), 2.44 (t, J=6.9 Hz, 2H), 2.27 (s, 3H), 1.15 (t, J=7.2 Hz, 3H). ¹³C NMR (100 MHz, CDCl₃): 195.8, 171.0, 148.5, 140.7, 140.0, 135.6, 126.6, 126.3, 124.0, 123.5, 43.9, 34.3, 30.4, 24.5, 13.1. ESIMS m/z 352.9 ([M+H]⁺).

### Example 16

### Alternative preparation of S-(3-((3-chloro-1-(pyridin-3-yl)-1H-pyrazol-4-yl)(ethyl)amino)-3-oxopropyl) ethanethioate (IIId)

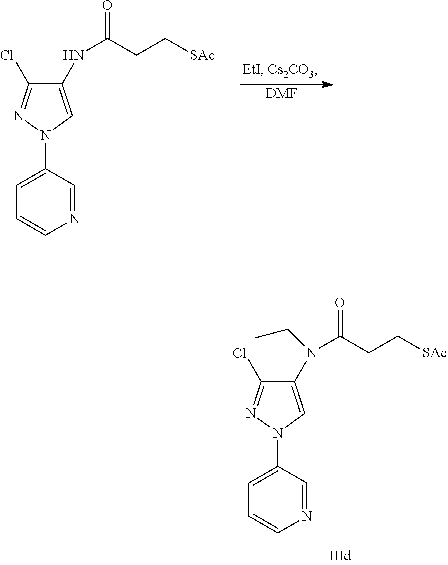

A 25-mL round bottom flask was charged with S-(3-((3-chloro-1-(pyridin-3-yl)-1H-pyrazol-4-yl)amino)-3-oxopropyl) ethanethioate (0.8 g, 2.46 mmol), DMF (5 mL), and cesium carbonate (1.85 g, 5.66 mmol) under nitrogen. To the suspension was added EtI (0.25 mL, 3.1 mmol) and the reaction was stirred at room temperature for 12 h. The reaction mixture was transferred to a separatory funnel containing water (25 mL) and extracted with EtOAc (3×50 mL) The combined organic layers were washed with brine (25 mL), dried over anhydrous Na₂SO₄, and concentrated under reduced pressure. The residue was purified twice by flash column chromatography (20-100% EtOAc/hexanes) to afford the desired product as an oil (0.38 g, 44% yield). ¹H NMR (400 MHz, CDCl₃): 8.95 (s, 1H), 8.61 (d, J=4.6 Hz, 1H), 8.05 (d, J=8.3 Hz, 1H), 7.99-7.88 (m, 1H), 7.45 (m, 1H), 3.70 (q, J=6.9 Hz, 2H), 3.10 (t, J=6.9 Hz, 2H), 2.44 (t, J=6.9 Hz, 2H), 2.27 (s, 3H), 1.15 (t, J=7.2 Hz, 3H). ESI-MS m/z 353.06 ([M+H]⁺).

### Example 17

### Preparation of N-(3-chloro-1-(pyridin-3-yl)-1H-pyrazol-4-yl)-N-ethyl-3-((3,3,3-trifluoropropyl)thio)propanamide (Vd)

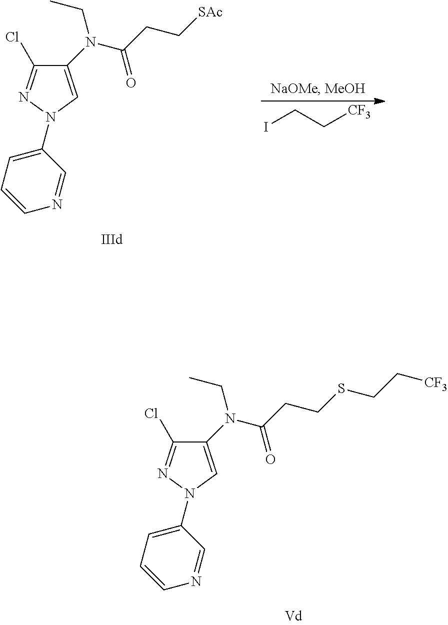

A 50-mL round bottom flask was charged with S-(3-((3-chloro-1-(pyridin-3-yl)-1H-pyrazol-4-yl)(ethyl)amino)-3-oxopropyl) ethanethioate (1.6 g, 4.54 mmol) and methanol (30 mL) The mixture was purged with a flow of nitrogen for 15 min. Sodium methoxide (0.735 g, 13.6 mmol) was added, and the suspension was stirred under nitrogen for 5 min. 1,1,1-Trifluoro-3-iodopropane (1.6 mL, 13 6 mmol) was added and the reaction was heated at 50° C. for 4 h, at which time HPLC analysis revealed complete conversion of S-(3-((3-chloro-1-(pyridin-3-yl)-1H-pyrazol-4-yl)(ethyl)amino)-3-oxopropyl) ethanethioate to product. The reaction was cooled to room temperature and transferred to a separatory funnel and EtOAc (100 mL) and water (50 mL) were added. The layers were separated and the aqueous phase was extracted with EtOAc (50 mL) The combined organic layers were washed with brine (25 mL), dried over anhydrous Na₂SO₄, and concentrated under reduced pressure. The residue was purified by flash column chromatography (20-100% EtOAc/hexanes) to afford an oil which solidified to give the desired product as a white solid (1.52 g, 82% yield, 97% HPLC purity). mp 79-80° C. ¹H NMR (400 MHz, CDCl₃): 8.94 (s, 1H), 8.62 (d, J=4.6 Hz, 1H), 8.04(d, J=9.3 Hz, 1H), 7.97 (s, 1H), 7.45 (m, 1H), 3.70 (q, J=7.0 Hz, 2H), 2.83 (t, J=7.2 Hz, 2H), 2.70-2.57 (m, 2H), 2.43 (t, J=7.2 Hz, 2H), 2.40-2.27 (m, 2H), 1.15 (t, J=7.1 Hz, 3H). ESIMS m/z 406.9 ([M+H]⁺).

### Example 18

### Alternative preparation of N-(3-chloro-1-(pyridin-3-yl)-1H-pyrazol-4-yl)-N-ethyl-3-((3,3,3-trifluoropropyl)thio)propanamide (Vd)

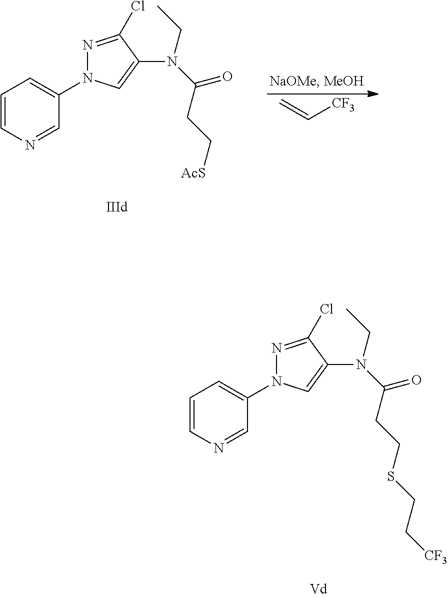

To a 200 mL flask was added IIId (0.61 g, 173 mmol) and dry methanol (7.2 g). The mixture was stirred under nitrogen and cooled to 5° C. NaOMe in methanol (25 wt %, 0.78 g, 2.09 equiv.) was added. A separate 50 mL Ace pressure tube was cooled in dry-ice and trifluoropropene (1.5 g) was condensed into the tube. The NaOMe reaction mixture was slowly transferred to the pressure tube containing trifluoropropene and the tube was sealed. The tube contents were stirred with a magnetic stir bar at 20° C. for 2 h. HPLC analysis showed complete conversion to product. The reaction mixture was diluted with saturated aq. NaCl solution (50 mL) and ethyl acetate (50 mL) and the organic layer was separated. The aqueous phase was extracted with additional ethyl acetate (50 mL). The organic phases were combined and concentrated to give a residue (0.49 g). The residue was loaded onto a column of silica gel (20 g) and eluted with 1:1 (hexanes/ethyl acetate) to 100% ethyl acetate. Product fractions were collected and evaporated to give the desired solid product (80 mg, (11%). Analytical data matched that of previously isolated product.

### Example 19

### Preparation of N-(3-chloro-1-(pyridin-3-yl)-1H-pyrazol-4-yl)-N-ethyl-3-mercaptopropanamide (IIId-1)

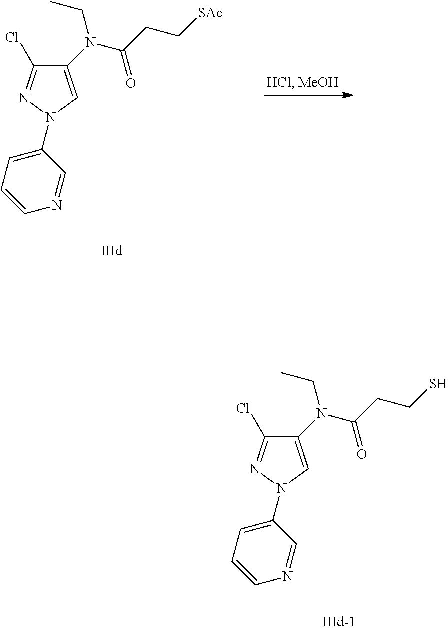

A 25-mL round bottom flask equipped with a magnetic stirrer was charged with methanol (35 mL) under nitrogen. The reaction was cooled to 0° C. and AcCl (10 mL, 140 mmol) was added dropwise over 15 min. A solution of S-(3-((3-chloro-1-(pyridin-3-yl)-1H-pyrazol-4-yl)(ethyl)amino)-3-oxopropyl) ethanethioate (1.7 g, 4.97 mmol) in MeOH (15 mL) was added to the above solution. The reaction was stirred for 2 h at 45° C., at which point HPLC analysis indicated complete conversion of S-(3-((3-chloro-1-(pyridin-3-yl)-1H-pyrazol-4-yl)(ethyl)amino)-3-oxopropyl) ethanethioate to the product. The reaction was concentrated under reduced pressure to -10 mL and water (100 mL) was added. Saturated aq. NaHCO₃ was added until pH 6, and the mixture was then transferred to a separatory funnel. The aqueous phase was extracted with EtOAc (3×100 mL) and the organics were washed with brine (25 mL), dried over anhydrous Na₂SO₄, and concentrated under reduced pressure to afford an oil which solidified to give a grey solid (1.5 g, 96% yield, 96% HPLC purity), mp 70-73° C. ¹H NMR (400 MHz, CDCl₃): 8.94 (s, 1H), 8.60 (d, J=4.6 Hz, 1H), 8.04 (d, J=8.3 Hz, 1H), 7.98 (s, 1H), 7.44 (m, 1H), 3.70 (q, J=7.0 Hz, 2H), 2.76 (q, J=7.2 Hz, 2H), 2.46 (t, J=6.7 Hz, 2H), 1.65 (t, J=8.4 Hz, 1H), 1.15 (t, J=7.1 Hz, 3H). ¹³C NMR (100 MHz, CDCl₃): 171.1, 148.8, 141.1, 140.1, 135.7, 126.5, 126.4, 124.2, 124.0, 44.1, 38.0, 20.1, 13.3. ESI MS m/z 311.0 ([M+H]⁺).

### Example 20

### Preparation of N-(3-chloro-1-(pyridin-3-yl)-1H-pyrazol-4-yl)-N-ethyl-3-((3,3,3-trifluoropropyl)thio)-propanamide (Vd)

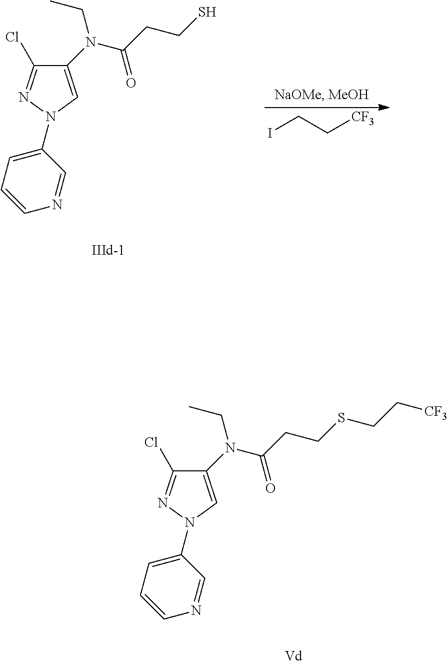 [SMILES: CCN(C(=O)CCS)c1cn(-c2cccnc2)nc1Cl ; FC(F)(F)CCI ; CCN(C(=O)CCSCCC(F)(F)F)c1cn(-c2cccnc2)nc1Cl]

A 50-mL round bottom flask equipped with a magnetic stirrer was charged with N-(3-chloro-1-(pyridin-3-yl)-1H-pyrazol-4-yl)-N-ethyl-3-mercaptopropanamide (1.1 g, 3.55 mmol) and DMF (8 mL) under nitrogen and stirred for 15 min. 1,1,1-Trifluoro-3-iodopropane (1.25 mL, 10.65 mmol) was added, followed by K₂CO₃ (1.47 g, 10.65 mmol). The reaction was heated at 50° C. for 4 h, at which time HPLC analysis indicated complete conversion of N-(3-chloro-1-(pyridin-3-yl)-1H-pyrazol-4-yl)-N-ethyl-3-mercaptopropanamide to the product. The reaction was cooled to room temperature and transferred to a separatory funnel and EtOAc (100 mL) and water (25 mL) were added and the aqueous phase was extracted with EtOAc (25 mL). The organic layers were washed with brine (25 mL), dried over anhydrous Na₂SO₄, and concentrated under reduced pressure. The residue was purified by flash column chromatography (30-90% EtOAc/hexanes) to afford the desired product as a white solid (1.2 g, 83% yield, 98% HPLC purity), mp 74-78° C. ¹H NMR (400 Hz, CDCl₃): 8.95 (s, 1H), 8.62 (d, J=4.6 Hz, 1H), (d, J=9.4 Hz, 1H), 7.97 (d, J=1.5 Hz, 1H), 7.45 (m, 1H), 3.71 (q, J=7.1 Hz, 2H), 2.83 (t, J=7.2 Hz, 2H), 2.71-2.57 (m, 2H), 2.43 (t, J=7.2 Hz, 2H), 2.39-2.24 (m, 2H), 1.15 (t, J=7.2 Hz, 3H). ESIMS m/z 406.9 ([M+H]⁺).

### Example 21

### Preparation of 3-chloro-N-(3-chloro-1H-pyrazol-4-yl)propanamide (IVa)

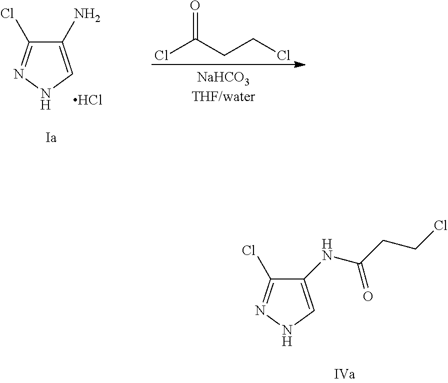 [SMILES: Nc1c[nH]nc1Cl ; O=C(Cl)CCCl ; Cl ; O=C(CCCl)Nc1c[nH]nc1Cl]

To a 250-mL, 3-neck flask was charged 3-chloro-1H-pyrazol-4-amine hydrochloride (10.01 g, 65.0 mmol), THF (50 mL), and water (50.0 mL) The resulting suspension was cooled to 5° C. and NaHCO₃ was added slowly, followed by dropwise addition of 3-chloropropanoyl chloride (7.5 g, 59.1 mmol) at <5° C. The reaction was stirred at <10° C. for 1 h, at which point TLC analysis (Eluent: 1:1 EtOAc/hexanes) indicated that the starting material was consumed and the desired product was formed. The reaction mixture was diluted with water (50 mL) and EtOAc (50 mL), and the layers were separated. The aqueous layer was extracted with EtOAc (20 mL), and the combined organic layers were concentrated to dryness to afford a white solid. The solid was dissolved in EtOAc (100 mL) at 60° C. to afford a clear solution. Hexane (150 mL) was added and the mixture was cooled to 20° C. The suspension was filtered and the solid was washed with hexanes (2×20 mL) to afford the desired product as a white solid (10.9 g, 88% yield). ¹H NMR (400 MHz, DMSO-d₆) δ 12.91 (s, 1H), 9.67 (s, 1H), 8.03 (d, 1.6 Hz, 1H), 3.85 (t, J=6.3 Hz, 2H), 2.85 (t, J=6.3 Hz, 2H). ¹³C NMR (126 MHz, DMSO-d₆) δ 166.99, 129.51, 123.04, 115.94, 40.21, 37.37. ESIMS m/z 208.0 ([M+H]⁺).

### Example 22

### Preparation of S-(3-((3-chloro-1H-pyrazol-4-yl)amino)-3-oxopropyl) ethanethioate (IIIa)

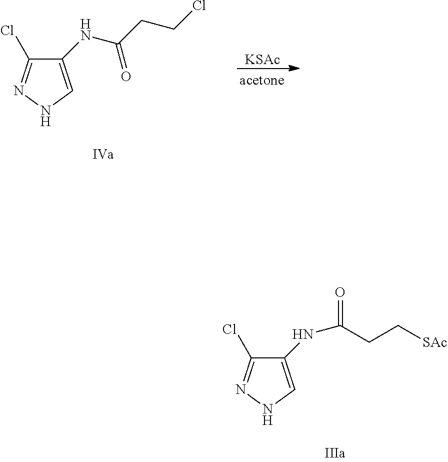

To a solution of 3-chloro-N-(3-chloro-1H-pyrazol-4-yl)propanamide (10 g, 48.1 mmol) in acetone (140 mL) was added KSAc (6.59 g, 57.7 mmol). The reaction was heated at 56° C. for 2 h, after which it was cooled to room temperature and water (150 mL) was added to give a clear solution. The mixture was concentrated under reduced pressure and the remaining aqueous layer was extracted with EtOAc (2×100 mL) The organic layer was washed with brine (2×30 mL), water (2×30 mL), and dried over anhydrous Na₂SO₄. The organic layer was concentrated and the crude product was purified by silica gel column chromatography eluting with 50-80% EtOAc/hexanes to afford the desired product, S-(3-((3-chloro-1H-pyrazol-4-yl)amino)-3-oxopropyl) ethanethioate (IIIa), as a white solid (6.2 g, 50.5% yield). ¹H NMR (400 MHz, DMSO-d₆) δ 12.89 (s, 1H), 9.58 (s, 1H), 8.00 (d, J=1.8 Hz, 1H), 3.05 (t, J=6.9 Hz, 2H), 2.64 (t, J=6.9 Hz, 2H), 2.33 (s, 3H). ESIMS m/z 248.0 ([M+H]⁺).

### Example 23

### Preparation of 3-chloro-N-(3-chloro-1H-pyrazol-4-yl)propanamide (IVb)

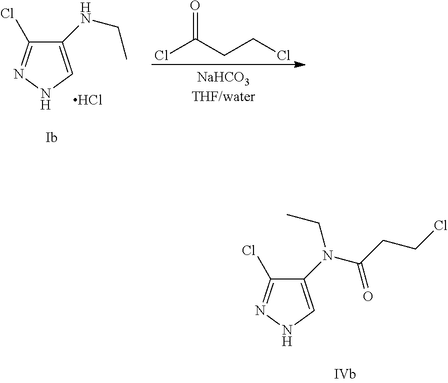

A 250-mL 3-neck flask was charged with 3-chloro-N-ethyl-1H-pyrazol-4-amine (7.1 g, 48.8 mmol), THF (50 mL), and water (50.0 mL) The resulting suspension was cooled to 5° C. and NaHCO₃ (7.45 g, 89 mmol) was added, followed by dropwise addition of 3-chloropropanoyl chloride (5.63 g, 44.3 mmol) at <5° C. The reaction was stirred at <10° C. for 1 h, at which point TLC (Eluent: 1:1 EtOAc/hexanes) analysis indicated the starting material was consumed and the desired product was formed. It was diluted with water (50 mL) and EtOAc (50 mL) and the layers separated. The aqueous layer was extracted with EtOAc (2×40 mL) and the combined organic layers were concentrated to dryness to afford a clear oil, which was purified by silica gel column chromatography using EtOAc/hexanes as eluent to afford the desired product, 3-chloro-N-(3-chloro-1H-pyrazol-4-yl)propanamide (IVb), as a white solid after drying (7.1 g, 67% yield), mp: 98-100° C. ¹H NMR (500 MHz, CDCl₃) δ 11.84 (s, 1H), 7.65 (s, 1H), 3.78 (t, J=6.7 Hz, 2H), 3.71 (q, J=7.2 Hz, 2H), 2.60 (t, J=6.8 Hz, 2H), 1.14 (t, J=7.2 Hz, 3H). ¹³C NMR (126 MHz, CDCl₃) δ 170.48, 138.15, 128.65, 120.72, 44.03, 39.82, 36.75, 12.97.

### Example 24

### Preparation of S-(3-((3-chloro-1H-pyrazol-4-yl)amino)-3-oxopropyl) ethanethioate (IIIb)

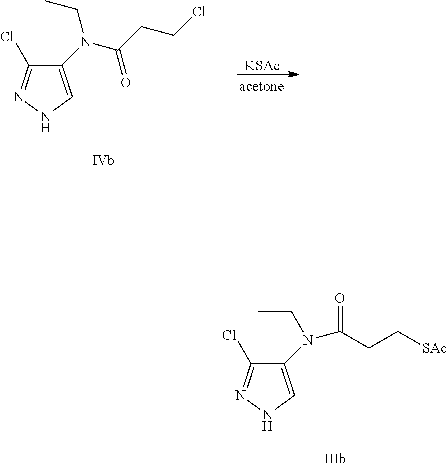

To a solution of 3-chloro-N-(3-chloro-1H-pyrazol-4-yl)-N-ethylpropanamide (6.4 g, 27.1 mmol) in acetone (200 mL) was added KSAc (3.71 g, 32.5 mmol). The reaction was heated at 56° C. for 2 h, after which it was cooled to room temperature and water (100 mL) was added to give a clear solution. The reaction mixture was concentrated to remove acetone and the remaining aqueous layer was extracted with EtOAc (3×30 mL). The organics were dried over anhydrous Na₂SO₄ filtered, and concentrated. The residue was purified by silica gel column chromatography using EtOAc/hexane as eluent to afford the desired product, S-(3-((3-chloro-1H-pyrazol-4-yl)amino)-3-oxopropyl) ethanethioate (IIIb), as a white solid after drying (3.8 g, 51% yield). ¹H NMR (400 MHz, CDCl₃): δ 11.91 (s, 1H), 7.60 (s, 1H), 3.66 (q, J=6.6 Hz, 2H), 3.09 (t, 6.8 Hz, 2H), 2.40 (t, J=6.7 Hz, 2H), 2.28 (s, 3H), 1.11 (t, J=7.1 Hz, 3H). ¹³C NMR (100 MHz, CDCl₃): δ 196.5, 171.8, 138.1, 128.6, 120.8, 44.1, 34.3, 30.7, 24.7, 13.1. ESIMS m/z 275.82 ([M+H]⁺).

### Example 25

### Preparation of 3-chloro-N-(3-chloro-1-(pyridin-3-yl)-1H-pyrazol-4-yl)propanamide (IVc)

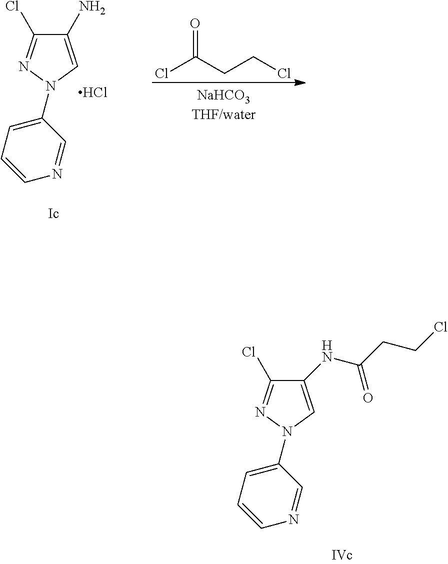 [SMILES: Nc1cn(-c2cccnc2)nc1Cl ; Cl ; O=C(Cl)CCCl ; O=C(CCCl)Nc1cn(-c2cccnc2)nc1Cl]

To a solution of 3-chloro-1-(pyridin-3-yl)-1H-pyrazol-4-amine (6.0 g, 30 8 mmol) in EtOAc (120 mL) was added NaHCO₃ (5.18 g, 61.7 mmol). The mixture was stirred at 20° C., and 3-chloropropanoyl chloride (3.24 mL, 33.9 mmol) was added over 10 min. The reaction mixture was stirred at 20° C. for 2 h and further stirred at 40° C. for 1 h, after which HPLC showed complete reaction. The reaction was cooled to 20° C. and diluted with EtOAc (200 mL). The solution was washed with water (2×40 mL), brine (2×30 mL) and dried over anhydrous Na₂SO₄ and filtered. The filtrate was concentrated to give the desired product, 3-chloro-N-(3-chloro-1-(pyridin-3-yl)-1H-pyrazol-4-yl)propanamide (IVc), as a white solid (8.8 g, 96% yield). ¹H NMR (400 MHz, CDCl₃) δ 8.98 (d, J=2.6 Hz, 1H), 8.66 (s, 1H), 8.56 (dd, 4.8, 1.4 Hz, 1H), 7.99 (ddd, J=8.3, 2.7, 1.4 Hz, 1H), 7.47-7.33 (m, 2H), 3.91 (t, J=6.3 Hz, 2H), 2.92 (t, J=6.3 Hz, 2H). ¹³C NMR (101 MHz, DMSO-d₆) δ 167.35, 146.95, 138.92, 134.91, 132.89, 124.96, 123.66, 121.90, 119.33, 40.09, 37.36. ESIMS m/z 285.0 ([M+H]⁺).

### Example 26

### Preparation of S-(3-((3-chloro-1-(pyridin-3-yl)-1H-pyrazol-4-yl)amino)-3-oxopropyl) ethanethioate (IIIc)

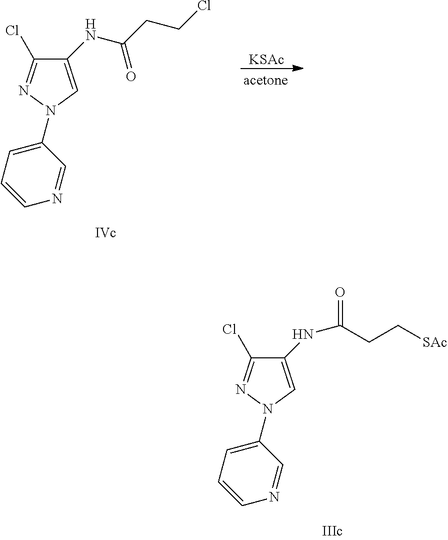

To a solution of 3-chloro-N-(3-chloro-1-(pyridin-3-yl)-1H-pyrazol-4-yl)propanamide (8.4 g, 29.5 mmol) in acetone (250 mL) was added KSAc (4.04 g, 35.4 mmol). The reaction was heated at 56° C. for 2 h, after which it was cooled and water (150 mL) was added to give a clear solution. The mixture was concentrated to give a white suspension. The suspension was filtered and the filter cake was rinsed with water (2×40 mL) The solid was dried under vacuum at 50° C. to afford the desired product, S-(3-((3-chloro-1-(pyridin-3-yl)-1H-pyrazol-4-yl)amino)-3-oxopropyl) ethanethioate (IIIc), as a white solid (9.2 g, 92% yield) mp 168-171° C. ¹H NMR (500 MHz, DMSO-d₆) δ 9.93 (s, 1H), 9.05 (dd, J=2.8, 0.7 Hz, 1H), 8.86 (s, 1H), 8.54 (dd, J=4.7, 1.4 Hz, 1H), 8.21 (ddd, J=8.4, 2.8, 1.5 Hz, 1H), 7.54 (ddd, J=8.4, 4.7, 0.8 Hz, 1H), 3.10(t, J=6.9 Hz, 2H), 2.73 (t, J=6.9 Hz, 2H), 2.34 (s, 3H). ¹³C NMR (101 MHz, DMSO-d₆) δ 194.71, 168.49, 146.91, 138.87, 134.89, 132.92, 124.92, 123.66, 121.86, 119.34, 34.16, 29.94, 23.62. ESIMS m/z ([M+H]⁺).

### Example 27

### Preparation of 3-chloro-N-(3-chloro-1-(pyridin-3-yl)-1H-pyrazol-4-yl)-N-ethylpropanamide (IVd)

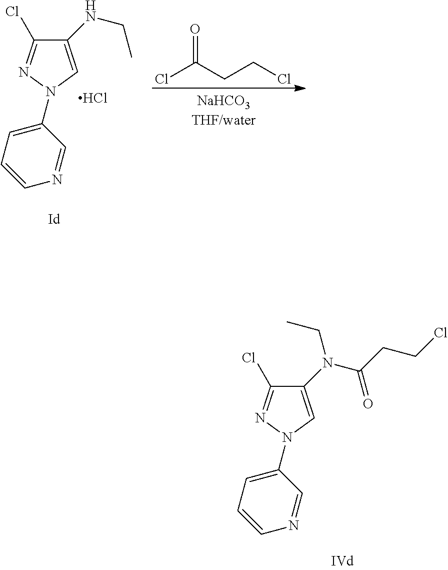

To a solution of 3-chloro-N-ethyl-1-(pyridin-3-yl)-1H-pyrazol-4-amine (6.1 g, 27.4 mmol) in EtOAc (120 mL) was added NaHCO₃ (4.60 g, 54.8 mmol). The mixture was stirred at 20° C. 3-Chloropropanoyl chloride (2.88 mL, 30.1 mmol) was added over 10 min. The reaction mixture was stirred at 20° C. for 2 h to give a brown gum. Water (40 mL) was added and the organic layer was separated. HPLC analysis indicated about 10% starting material remaining. The organic layer was dried over anhydrous Na₂SO₄ filtered and then NaHCO₃ (0.5 g) was added, followed by 3-chloropropanoyl chloride (0.3 mL). The mixture was further stirred for 1 h, at which point HPLC showed that starting material had been fully consumed. The reaction mixture was filtered through a filter paper and the filtrates were washed with water (2×40 mL), brine (2×30 mL), and dried over anhydrous Na₂SO₄. It was filtered and concentrated to the desired product, 3-chloro-N-(3-chloro-1-(pyridin-3-yl)-1H-pyrazol-4-yl)-N-ethylpropanamide (IVd), as a brown solid (8.6 g, 96% yield). ¹H NMR (400 MHz, CDCl₃) δ 8.97 (s, 1H), 8.64 (d, J=4.6 Hz, 1H), 8.06 (ddd, J=8.4, 2.8, 1.4 Hz, 1H), 7.99 (s,1H), 7.47 (dd, 8.4, 4.7 Hz, 1H), 3.77 (dt, J=22.8, 7.0 Hz, 4H), 2.64 (t, J=6.7 Hz, 2H), 1.18 (t, J=7.2 Hz, 3H). ¹³C NMR (101 MHz, CDCl₃) δ 169.83, 148.71, 140.88, 140.04, 135.60, 126.55, 126.34, 124.13, 123.61, 44.04, 39.85, 36.75, 13.10. ESIMS m/z 313.0 ([M+H]⁺).

### Example 28

### Preparation of S-(3-((3-chloro-1-(pyridin-3-yl)-1H-pyrazol-4-yl)(ethyl)amino)-3-oxopropyl) ethanethioate (IIId)

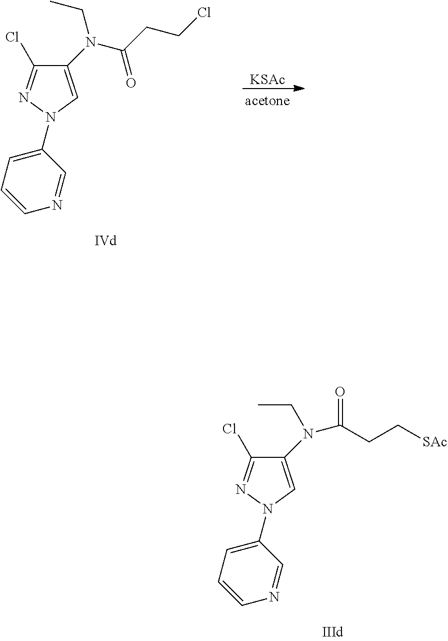

To a solution of 3-chloro-N-(3-chloro-1-(pyridin-3-yl)-1H-pyrazol-4-yl)-N-ethylpropanamide (8.3 g, 26.5 mmol) in acetone (110 mL) was added KSAc (3.63 g, 31.8 mmol). The reaction was heated at 56° C. for 2 h, after which it was cooled and poured into a separatory funnel containing water (100 mL) and EtOAc (100 mL). The layers were separated and the aqueous layer was extracted with EtOAc (3×25 mL). The combined organic extracts were dried over anhydrous Na₂SO₄, filtered and concentrated. The crude residue was purified via silica gel column chromatography (0-100% EtOAc/hexanes) to give a brown oil. ¹H NMR showed that ˜10-15% starting material remained and therefore the residue was dissolved in acetone (100 mL) and KSAc (0.6 g) was added. The mixture was heated at reflux for 3 h, after which the reaction was cooled to 20° C. and water (100 mL) was added to give a clear yellow solution. Acetone was evaporated under reduced pressure and the remaining mixture was extracted with EtOAc (2×100 mL). The organic layer was dried over anhydrous Na₂SO₄, filtered and concentrated to afford the desired product, S-(3-((3-chloro-1-(pyridin-3-yl)-1H-pyrazol-4-yl)(ethyl)amino)-3-oxopropyl) ethanethioate (IIId), as a brown oil (8.4 g, 86% yield). ¹H NMR (500 MHz, CDCl₃) δ 8.96 (d, J=2.6 Hz, 1H), 8.63 (dd, J=4.8,1.4 Hz, 1H),(ddd, J=8.3,2.8, 1.4 Hz, 1H), 7.96 (s, 1H), 7.47 (dt, J=8.3, 4.0 Hz, 1H), 3.71 (q, J=7.2 Hz,2H), 3.11 (t, 7.0 Hz, 2H), 2.45 (t, J=7.0 Hz, 2H), 2.28 (s, 3H), 1.17 (q, J=7.4 Hz, 3H). ¹³C NMR (126 MHz, CDCl₃) δ 195.99, 171.07, 148.67, 140.83, 140.09, 135.65, 126.42, 126.39, 124.09, 123.63, 43.93, 34.33, 30.53, 24.58, 13.13. ESIMS m/z 353.0 ([M+H]⁺).

What is claimed is:

1. A process for preparing a compound of the formula V 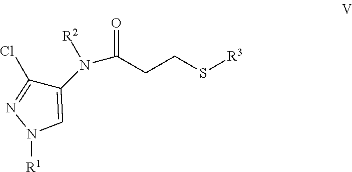
wherein R¹ is H or pyridin-3-yl; R² is H or C₁-C₆ alkyl; and R³ is C₁-C₆ alkyl optionally substituted with one or more halogen atoms or C₁-C₃ alkyl-C₃-C₆ cycloalkyl optionally substituted with one or more halogen atoms,
comprising
(a) contacting a compound of the formula I 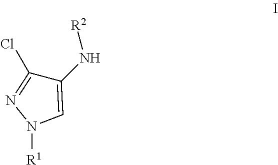
wherein R¹ is H or pyridin-3-yl; and R² is H or C₁-C₆ alkyl, with a compound of the formula X—C(O)CH═CH₂, wherein X in a leaving group, in the presence of a base and a solvent to provide a compound of the formula II 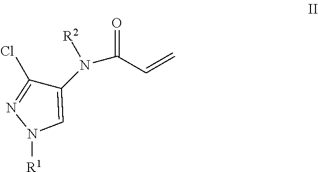
wherein R¹ is H or pyridin-3-yl; and R² is H or C₁-C₆ alkyl;
(b) contacting a compound of the formula II 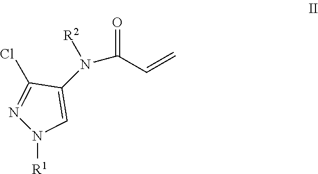
wherein R¹ is H or pyridin-3-yl; and R² is H or C₁-C₆ alkyl, with a thioacetate in the presence of an acid and a solvent to provide the compound of the formula III 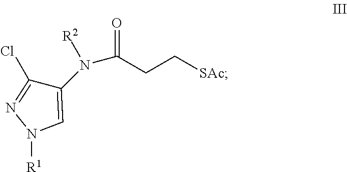
and
(c) contacting a compound of the formula III 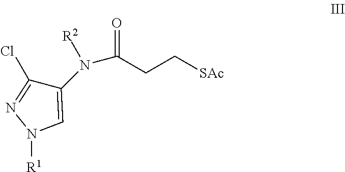
wherein R¹ is H or pyridin-3-yl; and R² is H or C₁-C₆ alkyl, with an alkylating agent in the presence of a base and a solvent to provide a compound of the formula V.

2. The process of claim 1, wherein X in the compound of the formula X—C(O)CH═CH₂, when present, is Cl, Br, I, —OC(O)C₁-C₆ alkyl or —OC(O)C₆-C₁₀ aryl.

3. The process of claim 1, wherein the base in step (a) is selected from the group consisting of sodium bicarbonate (NaHCO₃), sodium carbonate (Na₂CO₃), calcium carbonate (CaCO₃), cesium carbonate (Cs₂CO₃), lithium carbonate (Li₂CO₃), potassium carbonate (K₂CO₃), lithium hydroxide (LiOH), sodium hydroxide (NaOH), potassium hydroxide (KOH), cesium hydroxide (CsOH), calcium hydroxide (Ca(OH)₂), sodium diphosphate (Na₂HPO₄) and potassium phosphate (K₃PO₄).

4. The process of claim 1, wherein the solvent in step (a) is selected form the group consisting of diethyl ether, methylene dichloride (DCM), N,N-dimethylformamide (DMF), tetrahydrofuran (THF), ethyl acetate (EtOAc), acetone, acetonitrile (CH₃CN), and dimethylsulfoxide (DMSO).

5. The process of claim 1, wherein the alkylating agent of step (c) is a compound of the formula X¹-CH₂CH₂R³, wherein X¹ is a leaving group selected from the group consisting of Cl, Br, I, triflate (—OTf), tosylate (—OTs) and mesylate (—OMs), and R³ is C₁-C₆ alkyl optionally substituted with one or more halogen atoms or C₁-C₃ alkyl-C₃-C₆ cycloalkyl optionally substituted with one or more halogen atoms.

6. The process of claim 1, wherein the base in step (c) is selected from the group consisting of lithium hydroxide (LiOH), sodium hydroxide (NaOH), potassium hydroxide (KOH), cesium hydroxide (CsOH), calcium hydroxide (Ca(OH)₂), sodium hydride (NaH), lithium hydride (LiH), potassium hydride (KH), sodium methoxide (NaOCH₃) and sodium ethoxide (NaOCH₂CH₃).

7. The process of claim 1, wherein the thioacetate reagent in step (b) is of the formula MSAc, wherein M is H, Li, Na or K.

8. The process of claim 1, wherein the acid in step (b) is acetic acid, trifluoroacetic acid, p-toluenesulfonic acid, triflic acid or methanesulfonic acid.

9. A process comprising
(a) contacting a compound of the formula I 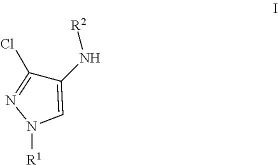
wherein R¹ is H or pyridin-3-yl; and R² is H or C₁-C₆ alkyl, with a compound of the formula X—C(O)CH═CH₂, wherein X in a leaving group, in the presence of a base and a solvent to provide a compound of the formula II 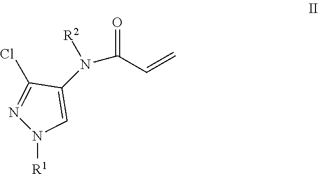
wherein R¹ is H or pyridin-3-yl; and R² is H or C₁-C₆ alkyl.

10. A process comprising
(b) contacting a compound of the formula II 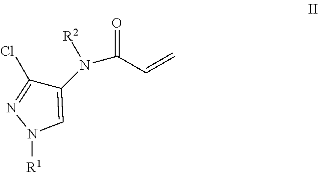
wherein R¹ is H or pyridin-3-yl; and R² is H or C₁-C₆ alkyl, with a thioacetate in the presence of an acid and a solvent to provide the compound of the formula III 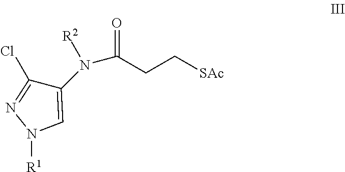
wherein R¹ is H or pyridin-3-yl; and R² is H or C₁-C₆ alkyl.

11. A process comprising
(c) contacting a compound of the formula III 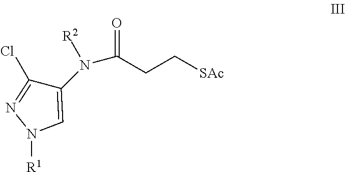
wherein R¹ is H or pyridin-3-yl; and R² is H or C₁-C₆ alkyl, with an alkylating agent in the presence of a base and a solvent to provide a compound of the formula V 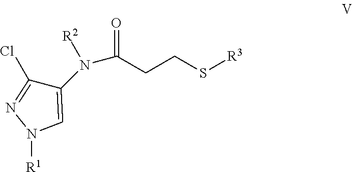
wherein R¹ is H or pyridin-3-yl; R² is H or C₁-C₆ alkyl; and R³ is C₁-C₆ alkyl optionally substituted with one or more halogen atoms or C₁-C₃ alkyl-C₃-C₆ cycloalkyl optionally substituted with one or more halogen atoms.

12. A process for preparing a compound of the formula V 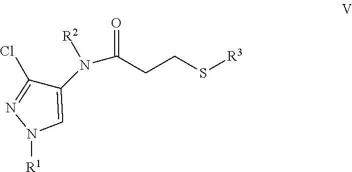
wherein R¹ is H or pyridin-3-yl; R² is H or C₁-C₆ alkyl; and R³ is C₁-C₆ alkyl optionally substituted with one or more halogen atoms or C₁-C₃ alkyl-C₃-C₆ cycloalkyl optionally substituted with one or more halogen atoms,
comprising
(a) contacting a compound of the formula I 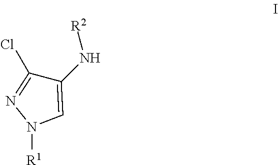
wherein R¹ is H or pyridin-3-yl; and R² is H or C₁-C₆ alkyl, with a compound of the formula X²—C(O)CH₂CH₂Y, wherein each of X² and Y is a leaving group, in the presence of a base and a solvent to provide a compound of the formula IV 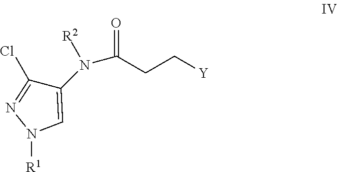
wherein R¹ is H or pyridin-3-yl; R² is H or C₁-C₆ alkyl, and Y is a leaving group;
(b) contacting a compound of the formula IV 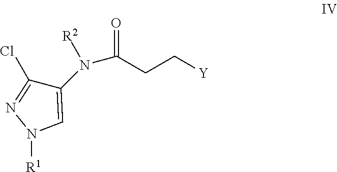
wherein R¹ is H or pyridin-3-yl; R² is H or C₁-C₆ alkyl; and Y is a leaving group with a thioacetate in the presence of a solvent to provide the compound of the formula III 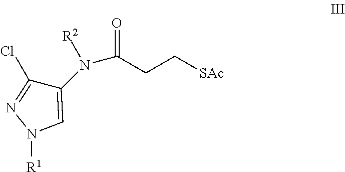
wherein R¹ is H or pyridin-3-yl; R² is H or C₁-C₆ alkyl; and
(c) contacting a compound of the formula III 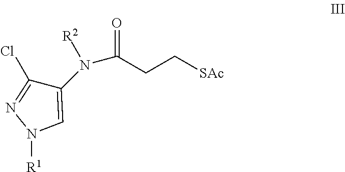
wherein R¹ is H or pyridin-3-yl; and R² is H or C₁-C₆ alkyl, with an alkylating agent in the presence of a base and a solvent to provide a compound of the formula V.

13. The process of claim 12, wherein X² is a leaving group selected from the group consisting of F, Cl, Br, I, —OC(O)C₁-C₆ alkyl or —OC(O)C₆-C₁₀ aryl, and Y is a leaving group selected from the group consisting of Cl, Br, I, triflate (—OTf), tosylate (—OTs) and mesylate (—OMs).

14. The process of claim 12, wherein the base in step (a) is selected from the group consisting of sodium bicarbonate (NaHCO₃), sodium carbonate (Na₂CO₃), calcium carbonate (CaCO₃), cesium carbonate (Cs₂CO₃), lithium carbonate (Li₂CO₃), potassium carbonate (K₂CO₃), lithium hydroxide (LiOH), sodium hydroxide (NaOH), potassium hydroxide (KOH), cesium hydroxide (CsOH), calcium hydroxide (Ca(OH)₂), sodium diphosphate (Na₂HPO₄) and potassium phosphate (K₃PO₄).

15. A process comprising
(a) contacting a compound of the formula I 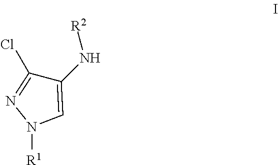
wherein R¹ is H or pyridin-3-yl; and R² is H or C₁-C₆ alkyl, with a compound of the formula X²—C(O)CH₂CH₂Y, wherein each of X² and Y is a leaving group, in the presence of a base and a solvent to provide a compound of the formula IV 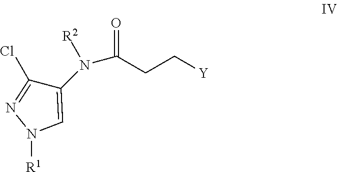
wherein R¹ is H or pyridin-3-yl; R² is H or C₁-C₆ alkyl, and Y is a leaving group.

16. A process comprising
(b) contacting a compound of the formula IV 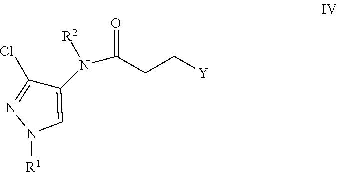
wherein R¹ is H or pyridin-3-yl; R² is H or C₁-C₆ alkyl; and Y is a leaving group with a thioacetate in the presence of a solvent to provide the compound of the formula III 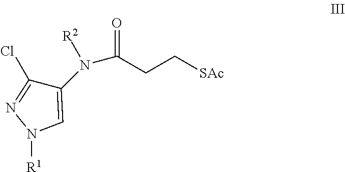
wherein R¹ is H or pyridin-3-yl; R² is H or C₁-C₆ alkyl.

17. A compound of the formula

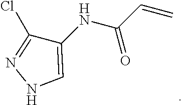

18. A compound of the formula

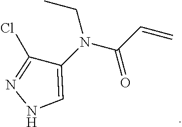

19. A compound of the formula

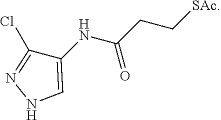

20. A compound of the formula

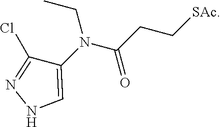

21. A compound of the formula [chemical structure]
